(19) United States
(12) Patent Application Publication
(10) Pub. No.: US 2013/0217644 A1
(43) Pub. Date: Aug. 22, 2013

(54) Pharmaceutical Compositions of 2'-C-Methyl-Guanosine, 5'-[2[(3-Hydroxy-2,2-Dimethyl-1-Oxopropyl)Thio]Ethyl N-(Phenylmethyl)Phosphoramidate]

(71) Applicant: IDENIX PHARMACEUTICALS, INC., (US)

(72) Inventors: Benjamin Alexander MAYES, Boston, MA (US); Adel M. MOUSSA, Burlington, MA (US); Rahela GASPARAC-KNEZIC, Watertown, MA (US); Alistair James STEWART, Lincoln, MA (US); Robert Vincent TUOHY, III, Philadelphia, PA (US); Dana Elaine MOSESON, Havertown, PA (US)

(73) Assignee: IDENIX PHARMACEUTICALS, INC., Cambridge, MA (US)

(21) Appl. No.: 13/763,545

(22) Filed: Feb. 8, 2013

Related U.S. Application Data
(60) Provisional application No. 61/598,274, filed on Feb. 13, 2012.

(51) Int. Cl. A61K 31/708

(52) U.S. Cl. CPC A61K 31/708
USPC 514/47

(57) ABSTRACT

Provided herein are pharmaceutical compositions, and in particular, oral pharmaceutical compositions, and methods of using these pharmaceutical compositions in the treatment of viral infections, including hepatitis C virus infections in hosts in need thereof. In certain embodiments, pharmaceutical compositions of 2′-C-methyl-guanosine, 5′-[2-[(3-hydroxy-2,2-dimethyl-1-oxopropyl)thio]ethyl N-(phenylmethyl)phosphoramidate] are provided which display remarkable efficacy and bioavailability for the treatment of, for example, HCV infection in a human.

### CROSS-REFERENCE TO RELATED APPLICATIONS

This application claims the benefit of U.S. application No. 61/598,274, filed on Feb. 13, 2012, which is incorporated by reference herein in its entirety, including any drawings.

### FIELD

Provided herein are oral pharmaceutical compositions and methods of using the pharmaceutical compositions in the treatment of viral infections, including hepatitis C virus infections in hosts in need thereof. In certain embodiments, pharmaceutical compositions of 2′-C-methyl-guanosine, 5′-[2-[(3-hydroxy-2,2-dimethyl-1-oxopropyl)thio]ethyl N-(phenylmethyl)phosphoramidate] are provided which display remarkable efficacy and exposure for the treatment of, for example, HCV infection in a human.

### BACKGROUND

Hepatitis C Virus

The hepatitis C virus (HCV) is the leading cause of chronic liver disease worldwide. (Boyer, N. et al., J. Hepatol. 32:98-112, 2000). HCV causes a slow growing viral infection and is the major cause of cirrhosis and hepatocellular carcinoma (Di Besceglie, A. M. and Bacon, B. R., Scientific American, October: 80-85, (1999); Boyer, N. et al., J. Hepatol. 32:98-112, 2000). An estimated 170 million persons are infected with HCV worldwide. (Boyer, N. et al., J. Hepatol. 32:98-112, 2000). Cirrhosis caused by chronic hepatitis C infection accounts for 8,000-12,000 deaths per year in the United States, and HCV infection is the leading indication for liver transplantation.

HCV is known to cause at least 80% of post transfusion hepatitis and a substantial proportion of sporadic acute hepatitis. Preliminary evidence also implicates HCV in many cases of “idiopathic” chronic hepatitis, “cryptogenic” cirrhosis, and probably hepatocellular carcinoma unrelated to other hepatitis viruses, such as Hepatitis B Virus (HBV). A small proportion of healthy persons appear to be chronic HCV carriers, varying with geography and other epidemiological factors. The numbers may substantially exceed those for HBV, though information is still preliminary; how many of these persons have subclinical chronic liver disease is unclear. (The Merck Manual, ch. 69, p. 901, 16th ed., (1992)).

A significant focus of current antiviral research is directed to the development of improved methods of treatment of chronic HCV infections in humans (Di Besceglie, A. M. and Bacon, B. R., Scientific American, October: 80-85, (1999)).

In light of the fact that HCV infection has reached epidemic levels worldwide, and has tragic effects on the infected patient, there remains a strong need to provide new effective pharmaceutical agents to treat hepatitis C that have low toxicity to the host. Further, given the rising threat of other flaviviridae infections, there remains a strong need to provide new effective pharmaceutical agents that have low toxicity to the host.

Therefore, there is a continuing need for effective treatments of flavivirus infections and HCV infections, and pharmaceutical compositions, particularly oral pharmaceutical compositions, for use in such treatments. Oral pharmaceutical compositions must meet a number of United States Food and Drug Administration (FDA) regulatory requirements for stability, manufacturability, and dissolution in order to be administered to a human or animal subject.

### SUMMARY

Provided herein are pharmaceutical compositions, and particularly oral pharmaceutical compositions, of 2′-C-methyl-guanosine, 5′-[2-[(3-hydroxy-2,2-dimethyl-1-oxopropyl)thio]ethyl N-(phenylmethyl)phosphoramidate], and methods useful for treating liver diseases such as HCV infection in a subject using such pharmaceutical compositions.

In certain embodiments, the oral pharmaceutical composition comprises one or members of the group consisting of 2′-C-methyl-guanosine, 5′-[2-[(3-hydroxy-2,2-dimethyl-1-oxopropyl)thio]ethyl N-(phenylmethyl)phosphoramidate], its pharmaceutically acceptable salts, its tautomers, its solvates, and its stereoisomers, in combination with one or more members of the group consisting of pharmaceutically acceptable excipients, carriers, and diluents, wherein when stored at 25° C. and 60% relative humidity for a duration of time of at least 12 months, the composition comprises not more than 15 μg of ethylene sulfide per each gram of compound 1 in the composition. In certain embodiments the oral pharmaceutical composition is a tablet.

In certain embodiments, the oral pharmaceutical composition is free from, or essentially free from, sodium lauryl sulfate, sodium stearyl fumarate, poloxamer 407, magnesium stearate, calcium phosphate dibasic dihydrate, phosphate dibasic anhydrous, calcium tribasic, sodium starch glycolate, and croscarmellose sodium. In certain embodiments, the oral pharmaceutical composition is a tablet including a disintegrant, a binder, and a tablet lubricant, and optionally, a flow aid.

In certain embodiments, the oral pharmaceutical composition is a tablet including crospovidone, hydrophobic colloidal silica, hydroxypropyl cellulose, poloxamer 188, silicified microcrystalline cellulose, and stearic acid. In certain embodiments, the oral pharmaceutical composition, which may be a tablet, comprises 25.0%±3.0% active pharmaceutical ingredient, 5.0%±2.0% crospovidone, 1.0%±0.5% hydrophobic colloidal silica, 5.0%±2.5% hydroxypropyl cellulose, 5.0%±2.0% poloxamer 188, 55.0%±7.0% silicified microcrystalline cellulose, and 2.0%±1.0% stearic acid; subject to the condition that the total does not exceed 100%, wherein the % represents weight %, and wherein the active pharmaceutical ingredient is 2′-C-methyl-guanosine, 5′-[2-[(3-hydroxy-2,2-dimethyl-1-oxopropyl)thio]ethyl N-(phenylmethyl)phosphoramidate], its pharmaceutically acceptable salts, its tautomers, its solvates, and its stereoisomers.

### DESCRIPTION OF EXEMPLARY EMBODIMENTS

Provided herein are pharmaceutical compositions, and particularly oral pharmaceutical compositions, of 2′-C-methyl-guanosine, 5′-[2-[(3-hydroxy-2,2-dimethyl-1-oxopropyl)thio]ethyl N-(phenylmethyl)phosphoramidate], and methods useful for treating liver diseases such as HCV infection in a subject using such oral pharmaceutical compositions.

### DEFINITIONS

When referring to the compositions and methods provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term herein, those in this section prevail unless stated otherwise.

Use of the term “herein” encompasses the specification, the abstract, and the claims of the present application.

Use of the singular herein includes the plural and vice versa unless expressly stated to be otherwise, or obvious from the context that such is not intended. That is, “a” and “the” refer to one or more of whatever the word modifies. For example, “a therapeutic agent” includes one therapeutic agent, two therapeutic agents, etc. Likewise, “the tablet” may refer to one, two or more tablets, and “the disease” may mean one disease or a plurality of diseases. By the same token, words such as, without limitation, “tablets” and “therapeutic agents” would refer to one tablet or therapeutic agent as well as to a plurality of tablets or therapeutic agents, unless, again, it is expressly stated or obvious from the context that such is not intended.

As used herein, unless specifically defined otherwise, any words of approximation such as without limitation, “about,” “essentially,” “substantially,” and the like mean that the element so modified need not be exactly what is described but can vary from the description. The extent to which the description may vary will depend on how great a change can be instituted and have one of ordinary skill in the art recognize the modified version as still having the properties, characteristics and capabilities of the unmodified word or phrase. In general, but with the preceding discussion in mind, a numerical value herein that is modified by a word of approximation may vary from the stated value by ±15%, unless expressly stated otherwise.

As used herein, any ranges presented are inclusive of the end-points. For example, “a temperature between 10° C. and 30° C.” or “a temperature from 10° C. to 30° C.” includes 10° C. and 30° C., as well as any specific temperature in between. Similarly, a temperature of 20° C.±10° C. would cover the same range as “a temperature between 10° C. and 30° C.”

As used herein, the use of “preferred,” “preferably,” or “more preferred,” and the like to modify an aspect of the invention refers to preferences as they existed at the time of filing of the patent application.

As used herein, when the phrase “a combination thereof,” or the phrase “any combination thereof” follows a list, the phrase refers to any combination of two or more items in the list. Likewise, the phrase “all combinations thereof” or “combinations thereof”, when following a list, refers to all combinations of two or more items in the list. The combinations may be of any or of all proportions. As an example, “A, B, C, and combinations thereof” would refer to “A, B, C, the combination of A and B, the combination of A and C, the combination of B and C, and the combination of A, B, and C,” where the combinations encompass all proportions. As used herein, a phrase such as “A, B, C, or any combination of A, B, and C” would be the same as “A, B, C, or any combination thereof” Analogously, the phrase “A, B, C, and combinations of A, B, and C” would be the same as “A, B, C, and combinations thereof”

“Pharmaceutically acceptable salt” refers to any salt of a compound described herein which retains its biological properties and which is not toxic or otherwise undesirable for pharmaceutical use. Such salts may be derived from a variety of organic and inorganic counter-ions well known in the art. Such salts include, but are not limited to: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) salts formed when an acidic proton present in the parent compound either (a) is replaced by a metal ion, e.g., an alkali metal ion, an alkaline earth ion or an aluminum ion, or alkali metal or alkaline earth metal hydroxides, such as sodium, potassium, calcium, magnesium, aluminum, lithium, zinc, and barium hydroxide, ammonia or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Pharmaceutically acceptable salts further include, by way of example only and without limitation, sodium, potassium, calcium, magnesium, ammonium, tetraalkylammonium and the like, and when the compound contains a basic functionality, salts of non-toxic organic or inorganic acids, such as hydrohalides, e.g. hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)-benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate and the like.

“Solvate” refers to a compound described herein or a salt thereof that further includes a stoichiometric or non-stoichiometric amount of solvent bound by non-covalent intermolecular forces. Where the solvent is water, the solvate is a hydrate.

The term “host”, as used herein, refers to any unicellular or multicellular organism in which the virus can replicate, including cell lines and animals, and in certain embodiments, a human. Alternatively, the host can be carrying a part of the Flaviviridae viral genome, whose replication or function can be altered by the compounds described herein. The term host specifically includes infected cells, cells transfected with all or part of the Flaviviridae genome and animals, in particular, primates (including chimpanzees) and humans. In most animal applications of the present invention, the host is a human patient. Veterinary applications, in certain indications, however, are clearly anticipated by the present invention (such as chimpanzees).

As used herein, the terms “subject” and “patient” are used interchangeably herein. The terms “subject” and “subjects” refer to an animal, such as a mammal including a non-primate (e.g., a cow, pig, horse, cat, dog, rat, and mouse) and a primate (e.g., a monkey such as a cynomolgous monkey, a chimpanzee and a human), and for example, a human. In certain embodiments, the subject is refractory or non-responsive to current treatments for hepatitis C infection. In another embodiment, the subject is a farm animal (e.g., a horse, a cow, a pig, etc.) or a companion animal (a.k.a. pet) (e.g., a dog or a cat). In certain embodiments, the subject is a human.

As used herein, the terms “therapeutic agent” and “therapeutic agents” refer to any agent(s) which can be used in the treatment or prevention of a disease or one or more symptoms thereof. In certain embodiments, the term “therapeutic agent” includes a compound as described herein. In certain embodiments, a therapeutic agent is an agent which is known to be useful for, or has been or is currently being used for the treatment or prevention of a disease or one or more symptoms thereof.

“Therapeutically effective amount” refers to an amount of a compound or composition that, when administered to a subject for treating a disease, is sufficient to effect such treatment for the disease. A “therapeutically effective amount” can vary depending on, inter alia, the compound, the disease and its severity, and the age, weight, etc., of the subject to be treated.

“Treating” or “treatment” of any disease refers, in certain embodiments, to ameliorating a disease that exists in a subject. In another embodiment, “treating” or “treatment” includes ameliorating at least one physical parameter, which may be indiscernible by the subject. In yet another embodiment, “treating” or “treatment” includes modulating the disease, either physically (e.g., stabilization of a discernible symptom) or physiologically (e.g., stabilization of a physical parameter) or both. In yet another embodiment, “treating” or “treatment” includes delaying the onset of the disease.

As used herein, the terms “prophylactic agent” and “prophylactic agents” as used refer to any agent(s) which can be used in the prevention of a disease or one or more symptoms thereof. In certain embodiments, the term “prophylactic agent” includes a compound described herein. In certain other embodiments, the term “prophylactic agent” does not refer a compound described herein. For example, a prophylactic agent is an agent which is known to be useful for, or has been or is currently being used to prevent or impede the onset, development, progression and/or severity of a disease.

As used herein, the phrase “prophylactically effective amount” refers to the amount of a therapy (e.g., prophylactic agent) which is sufficient to result in the prevention or reduction of the development, recurrence or onset of one or more symptoms associated with a disease, or to enhance or improve the prophylactic effect(s) of another therapy (e.g., another prophylactic agent).

A compound which is a “therapeutic agent” may also be a “prophylactic agent.” Typically, the “prophylactically effective amount” of the compound is less than the “therapeutically effective amount” of the compound. However, there may be some overlap between the “prophylactically effective amount” of a compound and the “therapeutically effective amount” of the compound.

As used herein, the phrase “grams of compound X” when used in reference to the quantity of compound X in a pharmaceutical composition refers to the grams of compound X, if present, in the composition in addition to the equivalent number of grams of compound X provided by all salts and solvates (including hydrates) of compound X, if present, in the composition.

As used herein, with reference to “particle size” when used in reference to a therapeutic agent (also known as “drug substance” and “active pharmaceutical ingredient”) refers to the average particle size, as determined by dynamic light scattering (DLS), also referred to as photo correlation spectroscopy. Dynamic light scattering determines the hydrodynamic diameter or the Stokes diameter based on diffusion measurements, and includes solvent associated with the particle. For non-spherical particles, the reported “diameter” is actually the effective diameter that is the diameter of a sphere with the equivalent hydrodynamic radius. This means hydrodynamic diameter obtained from DLS is close to the volume-average diameter. A non-limiting example of a method for determining average diameters is International Standards Organization (ISO) 13321.

Particles are generally polydisperse, i.e., not all the same size. One measure of polydispersity is the ratio D90/D10. D90 and D10 are the diameters representing the 90% and 10% percentiles of the particle size distribution. For example, D90 and D10 are the diameters below which 90% and 10% of the particles fall for a number average diameter, or 90% or 10% of the surface area of the particles falls for the surface area average diameter, and the like. As used herein when referring to the polydispersity of a therapeutic agent (also known as “drug substance” and “active pharmaceutical ingredient”), D90 and D10 are determined by dynamic light scattering, discussed above, unless expressly stated otherwise.

### Oral Pharmaceutical Compositions

Provided herein are pharmaceutical compositions, particularly oral pharmaceutical compositions, of 2′-C-methyl-guanosine, 5′-[2-[(3-hydroxy-2,2-dimethyl-1-oxopropyl)thio]ethyl N-(phenylmethyl)phosphoramidate], which is shown below:

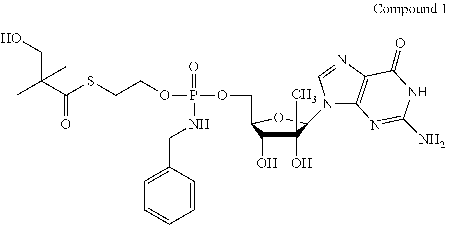

As used herein, the term “compound 1” refers to 2′-C-methyl-guanosine, 5′-[2-[(3-hydroxy-2,2-dimethyl-1-oxopropyl)thio]ethyl N-(phenylmethyl)phosphoramidate], shown above, and Compound 1 is also identified by the CAS Registry number (CAS number) 1036915-08-8, and the IUPAC name, 3-Hydroxy-2,2-dimethyl-thiopropionic acid S-(2-{[(2R,3R,4R,5R)-5-(2-amino-6-oxo-1,6-dihydro-purin-9-yl)-3,4-dihydroxy-4-methyl-tetrahydro-furan-2-ylmethoxy]-benzylamino-phosphoryloxy}-ethyl)ester. Compound 1 has also been referred to as Hydroxy-tBuSATE N-benzylphosphoramidate derivative of 2′-C-methylguanosine, and as IDX-14184 or IDX-184. As described in U.S. Pat. No. 7,951,789, which is incorporated by reference herein in its entirety, including any drawings, compound 1 has shown an ability to inhibit HCV replication in in-vitro assays. The pharmaceutical compositions provided herein avoid or limit the formation of ethylene sulfide, a potential degradant, contaminant, or both, of compound 1.

A pharmaceutical composition is formulated to be compatible with its intended route of administration. Examples of routes of administration include, but are not limited to, parenteral, e.g., intravenous, intradermal, subcutaneous, intramuscular, subcutaneous, oral, buccal, sublingual, inhalation, intranasal, transdermal, topical, transmucosal, intra-tumoral, intra-synovial and rectal administration.

Examples of dosage forms include, but are not limited to: tablets; caplets; capsules, such as soft elastic gelatin capsules and hard gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a subject, including suspensions (e.g., aqueous or non aqueous liquid suspensions, oil in water emulsions, or a water in oil liquid emulsions), solutions, and elixirs; liquid dosage forms suitable for parenteral administration to a subject; and sterile solids (e.g., crystalline or amorphous solids) that can be reconstituted to provide liquid dosage forms suitable for parenteral or oral administration to a subject.

With respect to the pharmaceutical compositions provided, the term “pharmaceutically acceptable” means approved by a regulatory agency of the United States Federal or a state government or listed in the United States Pharmacopeia (USP), United States National Formulary (NF), or other generally recognized pharmacopeia (such as and without limitation the European Pharmacopeia, the Japanese Pharmacopeia, and the British Pharmacopeia) for use in animals, and more particularly in humans.

The term “carrier” includes a diluent, adjuvant (e.g., Freund's adjuvant (complete and incomplete)), excipient, or vehicle with which the therapeutic agent is administered. Such pharmaceutical carriers can be sterile liquids, such as water and oils, including those of petroleum, animal, vegetable or synthetic origin, such as peanut oil, soybean oil, mineral oil, sesame oil and the like. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typical pharmaceutical compositions and dosage forms comprise one or more excipients in addition to an agent, also known as active pharmaceutical ingredient(s). The agent may be a therapeutic agent, a prophylactic agent, or both. Suitable excipients are well-known to those skilled in the art of pharmacy, and non limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific active pharmaceutical ingredients in the dosage form. The pharmaceutical composition or single unit dosage form, if desired, can also contain minor amounts of wetting or emulsifying agents, or pH buffering agents. In some cases vitamins, minerals, or other substances, which may have therapeutic uses, prophylactic uses, or both, themselves, may also be used as excipients. One of skill in the art can readily determine if a vitamin, mineral, or other substance is being used as an excipient in a pharmaceutical composition, and/or if the vitamin, mineral, or other substance is an agent in the pharmaceutical composition.

An oral pharmaceutical composition that was a liquid filled capsule was initially developed which exhibited an acceptable pharmacokinetic profile. The liquid filled capsule included low molecular weight polyethylene glycols, the active pharmaceutical ingredient (compound 1), and sodium lauryl sulfate, a solubilizer/surfactant. The initial liquid filled capsule composition, although exhibiting acceptable oral pharmacokinetic profile, exhibited unacceptable stability. Specifically, the level of ethylene sulfide in the capsules after three months of storage at 25° C. and 60% RH or under refrigerated conditions, 5° C., only allowed for a human clinical study of one month in duration.

A stable oral pharmaceutical composition, specifically a composition suitable for manufacturing tablets via direct compression without granulation, having bioequivalence (based on human data) to the previously used liquid filled capsule composition is provided herein. The oral pharmaceutical formulation exhibited no detectable levels of ethylene sulfide after nine months storage at 25° C. and 60% RH and at 5° C. and ambient humidity, and after six months storage at 40° C. and 75% RH (ICH and FDA storage conditions). This formulation was developed by careful selection of excipients to improve stability while still maintaining acceptable in-vivo pharmacokinetic profile. In addition, the tablets manufactured from the formulation exhibited low friability and good hardness, and reasonable content uniformity of the active pharmaceutical ingredient.

Provided herein are pharmaceutical compositions, particularly oral pharmaceutical compositions, of compound 1, its pharmaceutically acceptable salts, its tautomers, its solvates, or its stereoisomers, or any combination thereof, and one or more members of the group consisting of pharmaceutically acceptable excipients, carriers, and diluents, formulated such that the pharmaceutical composition, when stored at 25° C. and 60% relative humidity for a duration of time of at least 12 months, does not include ethylene sulfide at a level sufficient to exceed an intake of 1.5 μg ethylene sulfide per day when dosed as prescribed, or when dosed as needed. The therapeutic agent (or prophylactic agent or both) in a pharmaceutical compositions is conventionally referred to as the active pharmaceutical ingredient (API). As used herein, the term “compound 1 API” will refer to compound 1, its pharmaceutically acceptable salts, its tautomers, its solvates, or its stereoisomers, or any combination thereof.

Pharmaceutical compositions and single unit dosage forms provided herein may include a prophylactically effective amount, a therapeutically effective amount, or both, of compound 1, and typically one or more members of the group consisting of pharmaceutically acceptable carriers and excipients. In certain embodiments, a prophylactically effective amount, a therapeutically effective amount, or both, of compound 1 may comprise two or more single unit dosage forms.

In certain embodiments, the pharmaceutical composition is a pharmaceutical composition of compound 1 API, and the pharmaceutical composition includes not more than 1.5 μg of ethylene sulfide per daily dose of compound 1 in the pharmaceutical composition, whether a daily dose is a single or multiple dosage units, when stored at 25° C. and 60% relative humidity for a duration of time of at least 12 months.

Various non-limiting embodiments of the present invention are described in the following labeled paragraphs, paragraphs (A) to (AL):

(A) In certain embodiments, the pharmaceutical composition is a pharmaceutical composition of compound 1 API, and one or more members of the group consisting of pharmaceutically acceptable excipients, carriers, and diluents; where the pharmaceutical composition includes not more than 15 μg of ethylene sulfide per each gram of compound 1 in the pharmaceutical composition when stored at 25° C. and 60% relative humidity for a duration of time of at least 12 months. As used herein, the phrase “when stored at 25° C. and 60% relative humidity” refers to the industry standard for stability storage of pharmaceuticals as provided by the United States Food and Drug Administration (FDA) or the International Council on Harmonization of Technical Requirements for Registration of Pharmaceuticals for Human Use (ICH). As an example, the 25° C. and 60% RH storage condition as defined by the FDA guidance for industry specifies 25° C.±2° C. and 60% RH±5% RH.

(B) In certain embodiments, the pharmaceutical composition described herein, such as that described in paragraph (A), includes not more than 15 μg of ethylene sulfide per each gram of compound 1 in the pharmaceutical composition when stored at 25° C. and 60% relative humidity after a duration of time of at least 15 months.

(C) In certain embodiments, the pharmaceutical composition described herein, such as that described in paragraph (A), includes not more than 15 μg of ethylene sulfide per each gram of compound 1 in the pharmaceutical composition when stored at 25° C. and 60% relative humidity after a duration of time of at least 18 months.

(D) In certain embodiments, the pharmaceutical composition described herein, such as that described in paragraph (A), includes not more than 15 μg of ethylene sulfide per each gram of compound 1 in the pharmaceutical composition when stored at 25° C. and 60% relative humidity after a duration of time of at least 24 months.

(E) In certain embodiments, the pharmaceutical composition described herein, such as that described in paragraph (A), includes not more than 15 μg of ethylene sulfide per each gram of compound 1 in the pharmaceutical composition when stored at 25° C. and 60% relative humidity after a duration of time of at least 30 months.

(F) In certain embodiments, the pharmaceutical composition described herein, such as that described in paragraph (A), includes not more than 15 μg of ethylene sulfide per each gram of compound 1 in the pharmaceutical composition when stored at 25° C. and 60% relative humidity after a duration of time of at least 36 months.

(G) In certain embodiments, the pharmaceutical composition described herein, such as that of any one of paragraphs (A)-(F), includes not more than 13 μg of ethylene sulfide per each gram of compound 1 in the composition at the end of the storage duration.

(H) In certain embodiments, the pharmaceutical composition described herein, such as that of any one of paragraphs (A)-(F), includes not more than 10 μg of ethylene sulfide per each gram of compound 1 in the composition at the end of the storage duration.

(I) In certain embodiments, the pharmaceutical composition described herein, such as that of any one of paragraphs (A)-(F), includes not more than 8 μg of ethylene sulfide per each gram of compound 1 in the composition at the end of the storage duration.

(J) In certain embodiments, the pharmaceutical composition described herein, such as that of any one of paragraphs (A)-(F), includes not more than 5 μg of ethylene sulfide per each gram of compound 1 in the composition at the end of the storage duration.

(K) In certain embodiments, the pharmaceutical composition described herein, such as that of any one of paragraphs (A)-(F), includes not more than 3 μg of ethylene sulfide per each gram of compound 1 in the composition at the end of the storage duration.

(L) In certain embodiments, the pharmaceutical composition provided herein, such as that described in any one of paragraphs (A)-(K), includes compound 1 API in a micronized particle size range, that is the average particle size is less than or equal to 1000 μm, as determined by photon correlation spectroscopy.

(M) In certain embodiments, the pharmaceutical composition provided herein, such as that described in any one of paragraphs (A)-(K), includes compound 1 API of an average particle diameter in the range of 200 to 300 μm.

(N) In certain embodiments, the pharmaceutical composition provided herein, such as that described in any one of paragraphs (A)-(M), is lactose free.

(O) Further encompassed herein are anhydrous pharmaceutical compositions and dosage forms comprising compound 1 API. Pharmaceutical compositions and dosage forms, for example, tablets, provided herein, such as without limitation, those described in any one of paragraphs (A)-(N) above, can be prepared using anhydrous or low moisture containing ingredients and low moisture or low humidity conditions. Pharmaceutical compositions and dosage forms that comprise lactose and at least one active ingredient that comprise a primary or secondary amine can be anhydrous if substantial contact with moisture and/or humidity during manufacturing, packaging, and/or storage is expected. An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

(P) Further provided are pharmaceutical compositions and dosage forms, such as those described in paragraphs (A)-(O) above, that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

(Q) Further provided are pharmaceutical compositions and dosage forms, such as those described in paragraphs (A)-(P) above, that comprise, in addition to compound 1 API, a second agent, that is a therapeutic agent, a prophylactic agent, or both. Of course, the second agent will only be formulated with the compound 1 API, when, according to the judgment of those of skill in the art, such co-formulation should not unacceptably interfere with the activity of either therapeutic agent or the method of administration.

(R) In certain embodiments, the pharmaceutical composition described herein, such as any one of paragraphs (A)-(Q), is an oral pharmaceutical composition.

(S) In certain embodiments, the oral pharmaceutical composition described herein, such as that of paragraph (R), is a liquid composition for oral administration, of solutions which are pharmaceutically acceptable, suspensions, emulsions, syrups and elixirs containing inert diluents, such as water or liquid paraffin. In these compositions, the agent, compound 1 API and other optional agent(s), are typically mixed with one or more inert diluents, adjuvants, or both, such as sucrose, lactose, or starch.

(T) In certain embodiments, the oral pharmaceutical composition described herein, such as that of paragraph (R), is a solid oral composition such as and without limitation, tablets, pills, hard gelatin capsules, chewable tablets, caplets, powders or granules, or a combination thereof. These compositions can comprise one or members of the group consisting of binders, fillers, disintegrants, and lubricants, in addition to the therapeutic agent. These compositions may include a coating, which may be intended for controlled release, or which may be an aesthetic coating.

(U) In certain embodiments, the oral pharmaceutical composition described herein, such as that of paragraph (R), is a liquid filled capsule, such as a hard or soft gelatin capsule, filled with a liquid.

(V) In certain embodiments, the oral pharmaceutical composition provided herein, such as that described in paragraph (R), (T), or (U), is a single unit dosage form. In certain embodiments, the unit dosage comprises 1 to 1000 mg, 5 to 250 mg or 10 to 50 mg of compound 1. In particular embodiments, the unit dosages comprise about 1, 5, 10, 25, 50, 100, 125, 250, 500 or 1000 mg of compound 1.

(W) In certain embodiments, the oral pharmaceutical composition provided herein, such as that described in any one of paragraphs (R)-(V), is free from, or essentially free from, sodium lauryl sulfate, sodium stearyl fumarate, poloxamer 407, magnesium stearate, calcium phosphate dibasic dihydrate, phosphate dibasic anhydrous, calcium tribasic, sodium starch glycolate, and croscarmellose sodium.

(X) In certain embodiments, the oral pharmaceutical composition provided herein, such as that described in any one of paragraphs (R), (T), (V), and (W), is a tablet.

(Y) In certain embodiments, the oral pharmaceutical composition provided herein, such as that described in any one of paragraphs (R)-(X), includes a disintegrant. Disintegrants are used in the pharmaceutical compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active pharmaceutical ingredients should be used to form solid oral dosage forms. The amount of disintegrant used varies based upon the type of formulation, and is readily discernible to those of ordinary skill in the art. Typical pharmaceutical compositions, specifically tablets, comprise from about 0.5 to about 15 weight percent of disintegrant, specifically from about 1 to about 5 weight percent of disintegrant. Disintegrants that can be used in pharmaceutical compositions and dosage forms include, but are not limited to, agar, alginic acid, calcium carbonate, microcrystalline cellulose, croscarmellose sodium, crospovidone, polacrilin potassium, sodium starch glycolate, potato or tapioca starch, pre gelatinized starch, other starches, clays, other starches, other celluloses, gums, and mixtures thereof. Preferred disintegrants include, without limitation, crospovidone, pre gelatinized starch, and combinations thereof.

(Z) In certain embodiments, the oral pharmaceutical composition provided herein, such as that described in any one of paragraphs (R)-(Y), includes a binder. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre gelatinized starch, hydroxypropyl methyl cellulose, (e.g., Nos. 2208, 2906, 2910), microcrystalline cellulose, and mixtures thereof. Typical pharmaceutical compositions, specifically tablets, may comprise between about 0.5 and 20 weight percent a binder, with the range of 2 to 10 weight percent being more typical. Preferred binders include, without limitation, mannitol, microcrystalline cellulose, kaolin, soluble starch, sucrose, D-fructose, D-sorbitol, povidone (the USP name for poly(vinyl pyrrolidone)), hydroxypropyl cellulose, gelatin, and combinations thereof.

(AA) In certain embodiments, the oral pharmaceutical composition provided herein, such as that described in any one of paragraphs (R)-(Z), includes a filler. Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre gelatinized starch, and mixtures thereof. Some excipients may function as both a binder and a filler. The filler or filler/binder in pharmaceutical compositions is typically present in from about 50 to about 99 weight percent of the pharmaceutical composition or dosage form. Suitable forms of microcrystalline cellulose include, but are not limited to, the materials sold as AVICEL® PH 101, AVICEL® PH 103, AVICEL® RC 581, AVICEL® PH 105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL® RC 581. Suitable anhydrous or low moisture excipients or additives include AVICEL PH 103™ and Starch 1500 LM. Preferred fillers or filler/binders include, without limitation, microcrystalline cellulose, silicified microcrystalline cellulose, magnesium sulphate, mannitol, polyethylene glycol 8000, pre gelatinized starch, D-fructose, kaolin, soluble starch, sucrose, polyethylene glycol 3350, D-sorbitol, and combinations thereof.

(AB) In certain embodiments, the oral pharmaceutical composition provided herein, such as that described in any one of paragraphs (R)-(AA), includes a tablet lubricant. Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB 0 SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated. Preferred lubricants include, without limitation, stearic acid, castor oil, polyethylene glycol 8000, polyethylene glycol 3350, hydrogenated vegetable oil, and combinations thereof. Polyethylene glycol may be used as a binder, a filler, a lubricant, or a combination thereof depending upon the molecular weight. Low molecular weight polyethylene glycols that are liquids at room temperature and/or body temperature may also be used as a carrier or diluent.

(AC) In certain embodiments, the oral pharmaceutical composition provided herein, such as that described in any one of paragraphs (R)-(AB), includes a flow aid, which may also be referred to as a glidant. Non-limiting examples of flow aids include colloidal silica and hydrophobic colloidal silica.

(AD) In certain embodiments, the oral pharmaceutical composition provided herein, such as that described in any one of paragraphs (R)-(AC), includes, in addition to compound 1 API, crospovidone, hydrophobic colloidal silica, hydroxypropyl cellulose, poloxamer 188, silicified microcrystalline cellulose, and stearic acid. Crospovidone (CAS number 9003-39-8) is cross-linked poly(vinyl pyrrolidone). A preferred type of crospovidone is KOLLIDON® CL manufactured by BASF. KOLLIDON® CL is the standard particle size grade, and the vendor reports a volume average particle diameter of 90 to 130 μm as determined by laser light diffraction without solvent at 2 bar pressure (using the Malvern Mastersizer®). KOLLIDON® CL complies with the United States NF monograph, as well as the Ph. Eur. monograph. A preferred type of hydrophobic colloidal silica (CAS number 60842-32-2) is AEROSIL® R972, which complies with the United States NF and has a BET surface area of about 110±m²/gram. A preferred type of hydroxypropyl cellulose (CAS number 9000-64-2) is KLUCEL® EXF Pharm grade manufactured by Ashland Inc. The E grade is the lowest molecular weight provided by Ashland Inc. A 10% (by weight) aqueous solution of KLUCEL® EXF has a viscosity in the range of 300 to 600 cps when measured at 25° C.±0.5° C. using a Brookfield LVF, LVDV-1+, or LVDV-E viscometer, spindle 2, speed 30 rpm. The “X” refers to the fine grind particle size in which a minimum of 99.9% of the particles (by weight) pass through a U.S. 60 mesh sieve screen, a minimum of 90% pass-through a U.S. 80 mesh sieve screen, and a minimum of 80% passing through a U.S. 100 mesh sieve screen. A preferred type of poloxamer 188 (CAS number 9003-11-6) is LUTROL® 68 MICRO, a micronized poloxamer 188 manufactured by BASF. Poloxamers are block copolymers with a central block of polypropylene oxide) (PPO) having a block of poly(ethylene oxide) (PEO) on each side of the central PPO block where the PEO blocks are usually of the same length as determined by the number of ethylene oxide units. A preferred type of silicified microcrystalline cellulose (CAS numbers 9004-34-6 and 112945-52-5) is PROSOLV SMCC® 90 manufactured by JRS Pharma. PROSOLV SMCC® 90 complies with the United States NF monographs, and has an average particle size of about 110 microns as determined by laser diffraction. A preferred type of stearic acid is provided by Mallinckrodt, stearic acid (CAS number 57-11-4), powder, 2216.

(AE) In certain embodiments, the oral pharmaceutical composition provided herein, such as that described in paragraph (AD), exclusive of any exterior coating, consists essentially of compound 1 API, crospovidone, hydrophobic colloidal silica, hydroxypropyl cellulose, poloxamer 188, silicified microcrystalline cellulose, and stearic acid. It is understood that the active pharmaceutical ingredient, that is compound 1 API, may not assay at 100%. In other words, the active pharmaceutical ingredient “as received” can include impurities, potentially moisture, or a combination thereof.

(AF) In certain embodiments, at least 80.0 weight % of the oral pharmaceutical composition provided herein, such as that described in paragraph (AD), exclusive of any exterior coating, consists of the compound 1 API, crospovidone, hydrophobic colloidal silica, hydroxypropyl cellulose, poloxamer 188, silicified microcrystalline cellulose, and stearic acid.

(AG) In certain embodiments, at least 85.0 weight % of the oral pharmaceutical composition provided herein, such as that described in paragraph (AD), exclusive of any exterior coating, consists of compound 1 API, crospovidone, hydrophobic colloidal silica, hydroxypropyl cellulose, poloxamer 188, silicified microcrystalline cellulose, and stearic acid.

(AH) In certain embodiments, at least 90.0 weight % of the oral pharmaceutical composition provided herein, such as that described in paragraph (AD), exclusive of any exterior coating, consists of compound 1 API, crospovidone, hydrophobic colloidal silica, hydroxypropyl cellulose, poloxamer 188, silicified microcrystalline cellulose, and stearic acid.

(AI) In certain embodiments, at least 95.0 weight % of the oral pharmaceutical composition provided herein, such as that described in paragraph (AD), exclusive of any exterior coating, consists of compound 1 API, crospovidone, hydrophobic colloidal silica, hydroxypropyl cellulose, poloxamer 188, silicified microcrystalline cellulose, and stearic acid.

(AJ) In certain embodiments, the oral pharmaceutical composition provided herein, such as that in paragraph (AD), exclusive of any exterior coating, comprises 25.0%±3.0% active pharmaceutical ingredient, 5.0%±2.0% crospovidone, 1.0%±0.5% hydrophobic colloidal silica, 5.0%±2.5% hydroxypropyl cellulose, 5.0%±2.0% poloxamer 188, 55.0%±7.0% silicified microcrystalline cellulose, and 2.0%±1.0% stearic acid; subject to the condition that the total does not exceed 100%, and wherein the % represents weight %. As noted above, it is understood that the “compound 1 API” as received can include impurities and therefore, may not assay at 100%. As a result, the quantity of one or more excipients is adjusted to account for any changes in potency of the active pharmaceutical ingredient while maintaining a specific target tablet weight or composition weight. It is preferred, but not required, that the filler quantity be adjusted to account for a potency of less than 100% in the active pharmaceutical ingredient. It is also understood that the weight percent, for the excipients, is determined by the quantity of the materials added to the pharmaceutical formulation. Thus, the calculated % excipient can also include impurities, moisture, residual solvents, or a combination thereof included with the excipient as added to the pharmaceutical composition. For example if 25 kg of microcrystalline cellulose were to be added to a pharmaceutical composition of a total weight of 50 kg, the weight percent microcrystalline cellulose would be 50%, even if the microcrystalline cellulose contained 5 weight % water. The weight percent of compound 1 API, the active pharmaceutical ingredient, is determined by assay.

(AK) In certain embodiments, the tablet provided herein, such as any one described in paragraphs (X)-(AJ), has a hardness in the range of 10.0 to 13.5 kp (Kilopond) (test per USP general chapter <1217>), a friability of less than 0.5% (test per USP general chapter <1216>), a disintegration time of less than 10 minutes (test per USP general chapter <701>), and at least 75%, preferably 80%, of the label claim of active pharmaceutical ingredient is dissolved within 60 minutes as determined per USP apparatus II (paddle) at 75 rpm, 1000 ml of dissolution media of 4.5 acetate buffer (per USP) with 1% (by weight) sodium lauryl sulfate.

(AL) In certain embodiments, the oral pharmaceutical composition provided herein, such as that described in any one of paragraphs (R) and (T)-(AK), includes an exterior coating.

### Preparation of Compound 1

Compound 1 can be manufactured by methods known in the art. At least one method of preparing compound 1 is described in Example 3 of U.S. Pat. No. 7,951,789 which is incorporated by reference herein in its entirety, including any drawings.

### Manufacture of Oral Pharmaceutical Compositions

Typical oral dosage forms are prepared by combining the active pharmaceutical ingredient(s) in an intimate admixture with one or more members of the group consisting of excipients, adjuvants, and diluents, according to conventional pharmaceutical compounding techniques.

Because of their ease of administration, tablets and capsules represent the most advantageous oral dosage unit forms, in which case solid excipients are typically employed. If desired, tablets can be coated by standard aqueous or non-aqueous techniques. Such dosage forms can be prepared by any of the methods of pharmacy. In general, pharmaceutical compositions and dosage forms are prepared by uniformly and intimately admixing the active pharmaceutical ingredients with liquid carriers, finely divided solid carriers, or both, and then shaping the product into the desired presentation if necessary. Liquid carriers may be removed, or substantially removed, from the final dosage form.

For example, a tablet can be prepared by compression or molding. Compressed tablets can be prepared by compressing in a suitable machine, typically a rotary tablet press, the active pharmaceutical ingredients in a free flowing form such as powder or granules, optionally mixed with an excipient. Granules may be made by conventional granulation techniques including, without limitation, dry compaction, such as roller compaction followed by milling, high shear or low shear granulation, or fluid bed granulation. Low shear granulation, high shear granulation, and fluid bed granulation may use an aqueous or non-aqueous solvent or a melted excipient. Typically an aqueous or non-aqueous solvent is removed, or substantially removed, from the final pharmaceutical composition or dosage form. Molded tablets can be made by molding in a suitable machine a mixture of the powdered compound moistened with an inert liquid diluent. Exterior coating of tablets or other oral dosage forms may be accomplished by conventional tablet coating methods such as by using tablet coating equipment or a fluid bed with Wurster insert.

In certain embodiments, the oral pharmaceutical composition provided herein, such as that described in any one of paragraphs (R) and (T)-(AL), is a tablet prepared by blending and direct compression. In preferred embodiments, the tablet has a 200 mg target tablet weight, a 50 mg dose of compound 1, and is compressed utilizing 0.3125″ standard concave round tooling.

### Dosage and Unit Dosage Forms

In human therapeutics, the doctor will determine the posology which he considers most appropriate according to a preventive or curative treatment and according to the age, weight, stage of the infection and other factors specific to the subject to be treated. In certain embodiments, doses are from about 1 to about 1000 mg per day for an adult, or from about 5 to about 250 mg per day or from about 10 to 50 mg per day for an adult. In certain embodiments, doses are from about 5 to about 400 mg per day or 25 to 200 mg per day per adult. In certain embodiments, dose rates of from about 50 to about 500 mg per day are also contemplated. Doses above refer to the dose of compound 1 administered, or the dose of a second agent administered.

In further aspects, provided are methods of treating or preventing an HCV infection in a subject by administering, to a subject in need thereof, a pharmaceutical composition including an effective amount of compound 1 API, a second agent, or a combination thereof. The amount of the composition which will be effective in the prevention or treatment of a disease or one or more symptoms thereof will vary with the nature and severity of the disease or condition, and the route by which the active pharmaceutical ingredient is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, exemplary doses of compound 1, a second agent, or both included in a pharmaceutical composition including compound 1 API as described herein, such as any one of those described above in paragraphs (A)-(AL), include milligram or microgram amounts of compound 1, a second agent, or both, in the composition per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). For compositions provided herein, in certain embodiments, the dosage of compound 1, a second agent, or both administered to a subject is 0.140 mg/kg to 3 mg/kg of the subject's body weight, based on weight of the compound 1 in the composition. In certain embodiments, the dosage of compound 1, a second agent, or both, administered to a subject is between 0.20 mg/kg and 2.00 mg/kg, or between 0.30 mg/kg and 1.50 mg/kg of the subject's body weight.

In certain embodiments, the recommended daily dose range of a pharmaceutical composition provided herein, such as any one of those described above in paragraphs (A)-(AL), for the conditions described herein lie within the range of from about 0.1 mg to about 1000 mg per day, given as a single once-a-day dose or as divided doses throughout a day. In certain embodiments, the daily dose is administered twice daily in equally divided doses. In certain embodiments, a daily dose range should be from about 10 mg to about 200 mg per day, in other embodiments, between about 10 mg and about 150 mg per day, in further embodiments, between about 25 and about 100 mg per day. It may be necessary to use dosages of compound 1 outside the ranges disclosed herein in some cases, as will be apparent to those of ordinary skill in the art. Furthermore, it is noted that the clinician or treating physician will know how and when to interrupt, adjust, or terminate therapy in conjunction with subject response.

### Methods of Administration

In clinical practice therapeutic agents, such as without limitation compound 1 described herein, may be administered by any conventional route, in particular orally, parenterally, rectally or by inhalation (e.g. in the form of aerosols). In certain embodiments, pharmaceutical compositions provided herein are intended for oral administration.

Different therapeutically effective amounts may be applicable for different diseases and conditions, as will be readily known by those of ordinary skill in the art. Similarly, amounts sufficient to prevent, manage, treat or ameliorate such diseases and conditions, but insufficient to cause, or sufficient to reduce, adverse effects associated with the pharmaceutical composition provided herein are also encompassed by the above described dosage amounts and dose frequency schedules. Further, when a subject is administered multiple dosages of a pharmaceutical composition provided herein, not all of the dosages need be the same. For example, the dosage administered to the subject may be increased to improve the prophylactic or therapeutic effect of the pharmaceutical composition or it may be decreased to reduce one or more side effects that a particular subject is experiencing.

In certain embodiments, the dosage of the compound 1 in the pharmaceutical composition including compound 1 API provided herein, such as any one of those described above in paragraphs (A)-(AL), based on the mass of the compound 1 in the pharmaceutical composition, administered to prevent, treat, manage, or ameliorate a disease, or one or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of compound 1 in the pharmaceutical composition including compound 1 API provided herein, such as any one of those described above in paragraphs (A)-(AL), administered to prevent, treat, manage, or ameliorate a disease, or one or more symptoms thereof in a subject is a unit dose of 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

In certain embodiments, treatment or prevention can be initiated with one or more loading doses of compound 1 as provided by a pharmaceutical composition including compound 1 API as provided herein, such as any one of those described above in paragraphs (A)-(AL), followed by one or more maintenance doses. In such embodiments, the loading dose of compound 1 can be, for instance, about 60 to about 400 mg per day, or about 100 to about 200 mg per day for one day to five weeks. The loading dose of compound 1 can be followed by one or more maintenance doses of compound 1. In certain embodiments, each maintenance dose of compound 1 is, independently, about from about 10 mg to about 200 mg per day, between about 25 mg and about 150 mg per day, or between about 25 and about 80 mg per day. Maintenance doses of compound 1 can be administered daily and can be administered as single doses, or as divided doses.

Compound 1 is a prodrug and in-vivo converts to the 2′-C-methyl-guanosine monophosphate, which is in turn converted to the triphosphate in-vivo. The 2′-C-methyl-guanosine triphosphate is the active moiety. The triphosphate is also degraded in-vivo into the 2′-C-methyl-guanosine nucleoside. With respect to pharmacokinetic analyses, the 2′-C-methyl-guanosine nucleoside is one analytically measured species and can provide an indication of 2′-C-methyl-guanosine triphosphate level.

In certain embodiments, a dose of compound 1 in a pharmaceutical compositions including compound 1 API as provided herein, such as any one of those described above in paragraphs (A)-(AL), can be administered to achieve a steady-state concentration of 2′-C-methyl-guanosine in blood or serum of the subject. The steady-state concentration can be determined by measurement according to techniques available to those of skill or can be based on the physical characteristics of the subject such as height, weight and age. In certain embodiments, a sufficient amount of a pharmaceutical composition including compound 1 API as provided herein, such as any one of those described above in paragraphs (A)-(AL), is administered to achieve a steady-state concentration of 2′-C-methyl-guanosine in blood or serum of the subject of from about 0.1 to about 1000 ng/mL. In some embodiments, a sufficient quantity of a pharmaceutical composition including compound 1 API as provided herein, such as any one of those described above in paragraphs (A)-(AL), to provide loading doses of compound 1 can be administered to achieve steady-state blood or serum concentrations of 2′-C-methyl-guanosine of about 0.1 to about 1000 ng/mL for one to five days. In certain embodiments, maintenance doses of a pharmaceutical composition including compound 1 API as provided herein, such as any one of those described above in paragraphs (A)-(AL), can be administered to achieve a steady-state concentration of 2′-C-methyl-guanosine in blood or serum of the subject of from about 0.1 to about 1000 ng/mL.

In certain embodiments, administration of the same pharmaceutical composition, such as any one of those described in paragraphs (A)-(AL), may be repeated and the administrations may be separated by at least 1 day, 2 days, 3 days, 5 days, 10 days, 15 days, 30 days, 45 days, 2 months, 75 days, 3 months, or 6 months.

Provided herein are combination therapies, that is a therapy including administration of a pharmaceutical composition including compound 1 API provided herein, such as any one of those described in paragraphs (A)-(AL), and a second agent, the second agent being at least one member of the group consisting of therapeutic agents and prophylactic agents. The dosages of the second agents are to be used in the combination therapies provided herein. In certain embodiments, dosages lower than those which have been or are currently being used to prevent or treat HCV infection are used in the combination therapies provided herein. The recommended dosages of second agents can be obtained from the knowledge of those of skill. For those second agents that are approved for clinical use, recommended dosages are described in, for example, Hardman et al., eds., 1996, Goodman & Gilman's The Pharmacological Basis Of Basis Of Therapeutics 9^{th} Ed, Mc-Graw-Hill, New York; Physician's Desk Reference (PDR) 57^{th} Ed., 2003, Medical Economics Co., Inc., Montvale, N.J., which are incorporated herein by reference in their entirety. Exemplary second agents are described below.

In various embodiments, the therapies (e.g., a pharmaceutical composition including compound 1 API provided herein, such as any one of those described in paragraphs (A)-(AL), and a second agent) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In various embodiments, the therapies are administered no more than 24 hours apart or no more than 48 hours apart. In certain embodiments, two or more therapies are administered within the same patient visit. In other embodiments, the pharmaceutical composition including compound 1 API provided herein, such as any one of those described in paragraphs (A)-(AL), and the second agent are administered concurrently.

In other embodiments, the pharmaceutical composition including compound 1 API provided herein, such as any one of those described in paragraphs (A)-(AL), and the second agent are administered at about 2 to 4 days apart, at about 4 to 6 days apart, at about 1 week part, at about 1 to 2 weeks apart, or more than 2 weeks apart.

In certain embodiments, administration of the pharmaceutical composition including compound 1 API provided herein, such as any one of those described in paragraphs (A)-(AL), may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same pharmaceutical composition including compound 1 API provided herein, such as any one of those described in paragraphs (A)-(AL), may be repeated and the administration may be separated by at least at least 1 day, 2 days, 3 days, 5 days, 10 days, 15 days, 30 days, 45 days, 2 months, 75 days, 3 months, or 6 months.

In certain embodiments, a pharmaceutical composition including compound 1 API provided herein, such as any one of those described in paragraphs (A)-(AL), and a second agent are administered to a patient, for example, a mammal, such as a human, in a sequence and within a time interval such that compound 1 can act together with the other therapeutic agent to provide an increased benefit than if they were administered otherwise. For example, the second agent can be administered at the same time or sequentially in any order at different points in time; however, if not administered at the same time, they should be administered sufficiently close in time so as to provide the desired therapeutic or prophylactic effect. In certain embodiments, the pharmaceutical composition including compound 1 API provided herein, such as any one of those described in paragraphs (A)-(AL), and the second agent exert their effect at times which overlap. Each second agent can be administered separately, in any appropriate form and by any suitable route. In other embodiments, the pharmaceutical composition including compound 1 API provided herein, such as any one of those described in paragraphs (A)-(AL), is administered before, concurrently or after administration of the second agent.

In certain embodiments, the pharmaceutical composition including compound 1 API provided herein, such as any one of those described in paragraphs (A)-(AL), and the second agent are cyclically administered to a patient. Cycling therapy involves the administration of a first agent (e.g., a first prophylactic or therapeutic agent) for a period of time, followed by the administration of a second therapeutic agent and/or third therapeutic agent (e.g., a second and/or third prophylactic or therapeutic agents) for a period of time and repeating this sequential administration. Cycling therapy can reduce the development of resistance to one or more of the therapies, avoid or reduce the side effects of one of the therapies, and/or improve the efficacy of the treatment.

In certain embodiments, the pharmaceutical composition including compound 1 API provided herein, such as any one of those described in paragraphs (A)-(AL), and the second agent are administered in a cycle of less than about 3 weeks, about once every two weeks, about once every 10 days or about once every week. One cycle can comprise the administration of the pharmaceutical composition including compound 1 API provided herein, such as any one of those described in paragraphs (A)-(AL), orally once or twice daily, and the administration of the second agent by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

In other embodiments, courses of treatment are administered concurrently to a patient, i.e., individual doses of the second agent are administered separately yet within a time interval such that compound 1 can work together with the second agent. For example, one component can be administered once per week in combination with the other components that can be administered once every two weeks or once every three weeks. In other words, the dosing regimens are carried out concurrently even if the agents are not administered simultaneously or during the same day.

The second agent can act additively or synergistically with compound 1. In certain embodiments, compound 1 API is administered concurrently with one or more second agents in the same pharmaceutical composition. In another embodiment, a pharmaceutical composition including compound 1 API provided herein such as any one of those described in paragraphs (A)-(AL), is administered concurrently with one or more second agents in separate pharmaceutical compositions. In still another embodiment, a pharmaceutical composition including compound 1 API provided herein such as any one of those described in paragraphs (A)-(AL), is administered prior to or subsequent to administration of a second agent. Also contemplated are administration of a pharmaceutical composition including compound 1 API provided herein such as any one of those described in paragraphs (A)-(AL), and a second agent by the same or different routes of administration, e.g., oral and parenteral. In certain embodiments, when the pharmaceutical composition including compound 1 API provided herein, such as any one of those described in paragraphs (A)-(AL), is administered concurrently with a second agent that potentially produces adverse side effects including, but not limited to, toxicity, the second agent can advantageously be administered at a dose that falls below the threshold that the adverse side effect is elicited.

### Kits

Also provided are kits for use in methods of treatment of a liver disease such as HCV infections. The kits can include a pharmaceutical composition including compound 1 API provided herein such as any one of those described in paragraphs (A)-(AL), a second agent or composition, and instructions providing information to a health care provider regarding usage for treating the disease. Instructions may be provided in printed form or in the form of an electronic medium such as a floppy disc, CD, or DVD, or in the form of a website address where such instructions may be obtained. A pharmaceutical composition including compound 1 API provided herein, such as any one of those described in paragraphs (A)-(AL), that is a unit dose A, or a second agent or composition, can include a dosage such that when administered to a subject, a therapeutically or prophylactically effective plasma level of compound 1 can be maintained in the subject for at least 1 day. In some embodiments, a second agent or composition can be included as a sterile aqueous pharmaceutical composition or dry powder (e.g., lyophilized) composition.

In some embodiments, suitable packaging is provided. As used herein, “packaging” includes a solid matrix or material customarily used in a system and capable of holding within fixed limits a pharmaceutical composition including compound 1 API provided herein, such as any one of those described in paragraphs (A)-(AL), and/or a second agent suitable for administration to a subject. Such materials include glass and plastic (e.g., polyethylene, polypropylene, and polycarbonate) bottles, vials, paper, plastic, and plastic-foil laminated envelopes and the like. If electron beam sterilization techniques are employed, the packaging should have sufficiently low density to permit sterilization of the contents.

### Methods of Use

In certain embodiments, provided herein are methods for the treatment and/or prophylaxis of a host infected with Flaviviridae that includes the administration of an effective amount of a pharmaceutical composition including compound 1 API provided herein, such as any one of those described in paragraphs (A)-(AL). In certain embodiments, provided herein are methods for treating an HCV infection in a subject. In certain embodiments, the methods encompass the step of administering to the subject in need thereof a pharmaceutical composition including compound 1 API provided herein, such as any one of those described in paragraphs (A)-(AL), in combination with a second agent effective for the treatment or prevention of the infection. The pharmaceutical composition can be any pharmaceutical composition described herein, and the second agent can be any second agent described in the art or herein.

Flaviviridae that can be treated are discussed generally in Fields Virology, Editors: Fields, B. N., Knipe, D. M., and Howley, P. M., Lippincott-Raven Publishers, Philadelphia, Pa., Chapter 31, 1996. In a particular embodiment of the invention, the Flaviviridae is HCV. In an alternate embodiment of the invention, the Flaviviridae is a flavivirus or pestivirus. Specific flaviviruses include, without limitation: Absettarov, Alfuy, Apoi, Aroa, Bagaza, Banzi, Bouboui, Bussuquara, Cacipacore, Carey Island, Dakar bat, Dengue 1, Dengue 2, Dengue 3, Dengue 4, Edge Hill, Entebbe bat, Gadgets Gully, Hanzalova, Hypr, Ilheus, Israel turkey meningoencephalitis, Japanese encephalitis, Jugra, Jutiapa, Kadam, Karshi, Kedougou, Kokobera, Koutango, Kumlinge, Kunjin, Kyasanur Forest disease, Langat, Louping ill, Meaban, Modoc, Montana myotis leukoencephalitis, Murray valley encephalitis, Naranjal, Negishi, Ntaya, Omsk hemorrhagic fever, Phnom-Penh bat, Powassan, Rio Bravo, Rocio, Royal Farm, Russian spring-summer encephalitis, Saboya, St. Louis encephalitis, Sal Vieja, San Perlita, Saumarez Reef, Sepik, Sokuluk, Spondweni, Stratford, Tembusu, Tyuleniy, Uganda S, Usutu, Wesselsbron, West Nile, Yaounde, Yellow fever, and Zika.

Pestiviruses that can be treated are discussed generally in Fields Virology, Editors: Fields, B. N., Knipe, D. M., and Howley, P. M., Lippincott-Raven Publishers, Philadelphia, Pa., Chapter 33, 1996. Specific pestiviruses include, without limitation: bovine viral diarrhea virus (“BVDV”), classical swine fever virus (“CSFV,” also called hog cholera virus), and border disease virus (“BDV”).

In certain embodiments, the subject can be any subject infected with, or at risk for infection with, HCV. Infection or risk for infection can be determined according to any technique deemed suitable by the practitioner of skill in the art. In certain embodiments, subjects are humans infected with HCV.

In certain embodiments, the subject has never received therapy or prophylaxis for an HCV infection. In further embodiments, the subject has previously received therapy or prophylaxis for an HCV infection. For instance, in certain embodiments, the subject has not responded to an HCV therapy. For example, under current interferon therapy, up to 50% or more HCV subjects do not respond to therapy. In certain embodiments, the subject can be a subject that received therapy but continued to suffer from viral infection or one or more symptoms thereof. In certain embodiments, the subject can be a subject that received therapy but failed to achieve a sustained virologic response. In certain embodiments, the subject has received therapy for an HCV infection but has failed to show, for example, a 2 log₁₀ decline in HCV RNA levels after 12 weeks of therapy. It is believed that subjects who have not shown more than 2 log₁₀ reduction in serum HCV RNA after 12 weeks of therapy have a 97-100% chance of not responding.

In certain embodiments, the subject is a subject that discontinued an HCV therapy because of one or more adverse events associated with the therapy. In certain embodiments, the subject is a subject where current therapy is not indicated. For instance, certain therapies for HCV are associated with neuropsychiatric events. Interferon (IFN)-alfa plus ribavirin is associated with a high rate of depression. Depressive symptoms have been linked to a worse outcome in a number of medical disorders. Life-threatening or fatal neuropsychiatric events, including suicide, suicidal and homicidal ideation, depression, relapse of drug addiction/overdose, and aggressive behaviour have occurred in subjects with and without a previous psychiatric disorder during HCV therapy. Interferon-induced depression is a limitation for the treatment of chronic hepatitis C, especially for subjects with psychiatric disorders. Psychiatric side effects are common with interferon therapy and responsible for about 10% to 20% of discontinuations of current therapy for HCV infection.

Accordingly, provided are methods of treating or preventing an HCV infection in subjects where the risk of neuropsychiatric events, such as depression, contraindicates treatment with current HCV therapy. In certain embodiments, provided are methods of treating or preventing HCV infection in subjects where a neuropsychiatric event, such as depression, or risk of such indicates discontinuation of treatment with current HCV therapy. Further provided are methods of treating or preventing HCV infection in subjects where a neuropsychiatric event, such as depression, or risk of such indicates dose reduction of current HCV therapy.

Current therapy is also contraindicated in subjects that are hypersensitive to interferon or ribavirin, or both, or any other component of a pharmaceutical product for administration of interferon or ribavirin. Current therapy is not indicated in subjects with hemoglobinopathies (e.g., thalassemia major, sickle-cell anemia) and other subjects at risk from the hematologic side effects of current therapy. Common hematologic side effects include bone marrow suppression, neutropenia and thrombocytopenia. Furthermore, ribavirin is toxic to red blood cells and is associated with hemolysis. Accordingly, in certain embodiments, provided are methods of treating or preventing HCV infection in subjects hypersensitive to interferon or ribavirin, or both, subjects with a hemoglobinopathy, for instance thalassemia major subjects and sickle-cell anemia subjects, and other subjects at risk from the hematologic side effects of current therapy.

In certain embodiments, the subject has received an HCV therapy and discontinued that therapy prior to administration of a method provided herein. In further embodiments, the subject has received therapy and continues to receive that therapy along with administration of a method provided herein. The methods can be co-administered with other therapy for HCV according to the judgment of one of skill in the art. In certain embodiments, the methods or pharmaceutical compositions including compound 1 API provided herein, such as any one of those described in paragraphs (A)-(AL), can be co-administered with a reduced dose of the other therapy for HCV.

In certain embodiments, provided are methods of treating a subject that is refractory to treatment with interferon. For instance, in some embodiments, the subject can be a subject that has failed to respond to treatment with one or more agents selected from the group consisting of interferon, interferon α, pegylated interferon α, interferon plus ribavirin, interferon α plus ribavirin and pegylated interferon α plus ribavirin. In some embodiments, the subject can be a subject that has responded poorly to treatment with one or more agents selected from the group consisting of interferon, interferon α, pegylated interferon α, interferon plus ribavirin, interferon α plus ribavirin and pegylated interferon α plus ribavirin. In the above embodiments, a pro-drug form of ribavirin, such as taribavirin, may also have been used.

In certain embodiments, the subject has, or is at risk for, co-infection of HCV with HIV. For instance, in the United States, 30% of HIV subjects are co-infected with HCV and evidence indicates that people infected with HIV have a much more rapid course of their hepatitis C infection. Maier and Wu, 2002, World J Gastroenterol 8:577-57. The methods provided herein can be used to treat or prevent HCV infection in such subjects. It is believed that elimination of HCV in these subjects will lower mortality due to end-stage liver disease. Indeed, the risk of progressive liver disease is higher in subjects with severe AIDS-defining immunodeficiency than in those without. See, e.g., Lesens et al., 1999, J Infect Dis 179:1254-1258. In certain embodiments, compounds provided herein have been shown to suppress HIV in HIV subjects. Thus, in certain embodiments, provided are methods of treating or preventing HIV infection and HCV infection in subjects in need thereof.

In certain embodiments, the pharmaceutical compositions including compound 1 API provided herein, such as those described in paragraphs (A)-(AL), are administered to a subject following liver transplant. Hepatitis C is a leading cause of liver transplantation in the U.S., and many subjects that undergo liver transplantation remain HCV positive following transplantation. In certain embodiments, provided are methods of treating such recurrent HCV subjects with a compound or composition provided herein. In certain embodiments, provided are methods of treating a subject before, during or following liver transplant to prevent recurrent HCV infection.

### Assay Methods

Compounds, such as compound 1, can be assayed for HCV activity according to any assay known to those of skill in the art.

Further, compounds, such as compound 1, can be assayed for accumulation in liver cells of a subject according to any assay known to those of skill in the art. In certain embodiments, a compound can be administered to the subject, and a liver cell of the subject can be assayed for the compound or a derivative thereof, e.g. a nucleoside, nucleoside phosphate or nucleoside triphosphate derivative thereof.

In certain embodiments, compound 1 is administered to cells, such as liver cells, in vivo or in vitro, and the nucleoside triphosphate levels delivered intracellularly are measured, to indicate delivery of the compound and triphosphorylation in the cell. The levels of intracellular nucleoside triphosphate can be measured using analytical techniques known in the art. Methods of detecting ddATP are described herein below by way of example, but other nucleoside triphosphates can be readily detected using the appropriate controls, calibration samples and assay techniques.

In certain embodiments, ddATP concentrations are measured in a sample by comparison to calibration standards made from control samples. The ddATP concentrations in a sample can be measured using an analytical method such as HPLC, GC, and/or MS (high pressure liquid chromatography, gas chromatography, mass spectroscopy). In certain embodiments, a test sample is compared to a calibration curve created with known concentrations of ddATP to thereby obtain the concentration of that sample.

In certain embodiments, the samples are manipulated to remove impurities such as salts (Na⁺, K⁺, etc.) before analysis. In certain embodiments, the lower limit of quantitation is about ˜0.2 pmol/mL for hepatocyte cellular extracts particularly where reduced salt is present.

In certain embodiments, the method allows successfully measuring triphosphate nucleotides formed at levels of 1-10,000 pmol per million cells in e.g. cultured hepatocytes and HepG2 cells.

### Second Therapeutic Agents

In certain embodiments, the pharmaceutical compositions including compound 1 API provided herein, such as those described in paragraphs (A)-(AL), are useful in methods of treatment of a liver disease, that comprises further administration of a second agent effective for the treatment of the disease, such as HCV infection in a subject in need thereof. The second agent can be any agent known to those of skill in the art to be effective for the treatment of the disease, including those currently approved by the FDA.

In certain embodiments, a pharmaceutical composition including compound 1 API provided herein, such as those described in paragraphs (A)-(AL), is administered in combination with one second agent. In further embodiments, a second agent is administered in combination with a third agent. In still further embodiments, a second agent is administered in combination with two or more additional (third, fourth, etc.) agents.

As used herein, the term “in combination” includes the use of more than one therapy (e.g., one or more prophylactic and/or therapeutic agents). The use of the term “in combination” does not restrict the order in which therapies (e.g., prophylactic and/or therapeutic agents) are administered to a subject with a disease. A first therapy (e.g., a prophylactic or therapeutic agent such as compound 1 API included in a pharmaceutical composition provided herein, such as those described in paragraphs (A)-(AL)) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to a subject with a disease.

As used herein, the term “synergistic” includes a combination of a pharmaceutical composition including compound 1 API as provided herein, such as those described in paragraphs (A)-(AL), and another therapy (e.g., a prophylactic or therapeutic agent) which has been or is currently being used to prevent, manage or treat a disease, which is more effective than the additive effects of the therapies. A synergistic effect of a combination of therapies (e.g., a combination of prophylactic or therapeutic agents) permits the use of lower dosages of one or more of the therapies and/or less frequent administration of said therapies to a subject with a disease. The ability to utilize lower dosages of a therapy (e.g., a prophylactic or therapeutic agent) and/or to administer said therapy less frequently reduces the toxicity associated with the administration of said therapy to a subject without reducing the efficacy of said therapy in the prevention or treatment of a disease). In addition, a synergistic effect can result in improved efficacy of agents in the prevention or treatment of a disease. Finally, a synergistic effect of a combination of therapies (e.g., a combination of prophylactic or therapeutic agents) may avoid or reduce adverse or unwanted side effects associated with the use of either therapy alone.

The pharmaceutical compositions including compound 1 API provided herein, such as those described in paragraphs (A)-(AL), can be administered in combination or alternation with another therapeutic agent, in particular an anti-HCV agent. In combination therapy, effective dosages of two or more agents are administered together, whereas in alternation or sequential-step therapy, an effective dosage of each agent is administered serially or sequentially. The dosages given will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

It has been recognized that drug-resistant variants of flaviviruses, pestiviruses or HCV can emerge after prolonged treatment with an antiviral agent. Drug resistance most typically occurs by mutation of a gene that encodes for an enzyme used in viral replication. The efficacy of a drug against the viral infection can be prolonged, augmented, or restored by administering the compound in combination or alternation with a second, and perhaps third, antiviral compound that induces a different mutation from that caused by the principle drug. Alternatively, the pharmacokinetics, biodistribution or other parameter of the drug can be altered by such combination or alternation therapy. In general, combination therapy is typically preferred over alternation therapy because it induces multiple simultaneous stresses on the virus.

Any of the viral treatments described in the Background of the Invention can be used in combination or alternation with the pharmaceutical compositions including compound 1 API described in this specification, such as those described in paragraphs (A)-(AL). Nonlimiting examples of second agents include:

HCV Protease inhibitors: Examples include Medivir HCV Protease Inhibitor (HCV-PI or TMC435) (Medivir/Tibotec); MK-7009 (Merck), RG7227 (ITMN-191) (Roche/Pharmasset/InterMune), boceprevir (Victrelis™) (Merck), SCH 446211 (Merck), narlaprevir SCH900518 (Merck), ABT-450 (Abbott/Enanta), ACH-1625 (Achillion), BI 201335 (Boehringer Ingelheim), PHX1766 (Phenomix), VX-500 (Vertex), telaprevir (Incivek™) (Vertex), ACH-1625 (Achillion), and ACH-2684 (Achillion). Further examples of protease inhibitors include substrate-based NS3 protease inhibitors (Attwood et al., Antiviral peptide derivatives, PCT WO 98/22496, 1998; Attwood et al., Antiviral Chemistry and Chemotherapy 1999, 10, 259-273; Attwood et al., Preparation and use of amino acid derivatives as anti-viral agents, German Patent Pub. DE 19914474; Tung et al., Inhibitors of serine proteases, particularly hepatitis C virus NS3 protease, PCT WO 98/17679), including alphaketoamides and hydrazinoureas, and inhibitors that terminate in an electrophile such as a boronic acid or phosphonate (Llinas-Brunet et al, Hepatitis C inhibitor peptide analogues, PCT WO 99/07734); Non-substrate-based NS3 protease inhibitors such as 2,4,6-trihydroxy-3-nitro-benzamide derivatives (Sudo K. et al., Biochemical and Biophysical Research Communications, 1997, 238, 643-647; Sudo K. et al., Antiviral Chemistry and Chemotherapy, 1998, 9, 186), including RD3-4082 and RD3-4078, the former substituted on the amide with a 14 carbon chain and the latter processing a para-phenoxyphenyl group; and Sch 68631, a phenanthrenequinone, an HCV protease inhibitor (Chu M. et al., Tetrahedron Letters 37:7229-7232, 1996).

SCH 351633, isolated from the fungus Penicillium griseofulvum, was identified as a protease inhibitor (Chu M. et al., Bioorganic and Medicinal Chemistry Letters 9:1949-1952). Eglin c, isolated from leech, is a potent inhibitor of several serine proteases such as S. griseus proteases A and B, α-chymotrypsin, chymase and subtilisin. Qasim M. A. et al., Biochemistry 36:1598-1607, 1997.

U.S. patents disclosing protease inhibitors for the treatment of HCV include, for example, U.S. Pat. No. 6,004,933 to Spruce et al., which discloses a class of cysteine protease inhibitors for inhibiting HCV endopeptidase 2; U.S. Pat. No. 5,990,276 to Zhang et al., which discloses synthetic inhibitors of hepatitis C virus NS3 protease; U.S. Pat. No. 5,538,865 to Reyes et a; WO 02/008251 to Corvas International, Inc, and U.S. Pat. No. 7,169,760, US2005/176648, WO 02/08187 and WO 02/008256 to Schering Corporation. HCV inhibitor tripeptides are disclosed in U.S. Pat. Nos. 6,534,523, 6,410,531, and 6,420,380 to Boehringer Ingelheim and WO 02/060926 to Bristol Myers Squibb. Diaryl peptides as NS3 serine protease inhibitors of HCV are disclosed in WO 02/48172 and U.S. Pat. No. 6,911,428 to Schering Corporation. Imidazoleidinones as NS3 serine protease inhibitors of HCV are disclosed in WO 02/08198 and U.S. Pat. No. 6,838,475 to Schering Corporation and WO 02/48157 and U.S. Pat. No. 6,727,366 to Bristol Myers Squibb. WO 98/17679 and U.S. Pat. No. 6,265,380 to Vertex Pharmaceuticals and WO 02/48116 and U.S. Pat. No. 6,653,295 to Bristol Myers Squibb also disclose HCV protease inhibitors. Further examples of HCV serine protease inhibitors are provided in U.S. Pat. No. 6,872,805 (Bristol-Myers Squibb); WO 2006000085 (Boehringer Ingelheim); U.S. Pat. No. 7,208,600 (Vertex); US 2006/0046956 (Schering-Plough); WO 2007/001406 (Chiron); US 2005/0153877; WO 2006/119061 (Merck); WO 00/09543 (Boehringer Ingelheim), U.S. Pat. No. 6,323,180 (Boehringer Ingelheim) WO 03/064456 (Boehringer Ingelheim), U.S. Pat. No. 6,642,204 (Boehringer Ingelheim), WO 03/064416 (Boehringer Ingelheim), U.S. Pat. No. 7,091,184 (Boehringer Ingelheim), WO 03/053349 (Bristol-Myers Squibb), U.S. Pat. No. 6,867,185, WO 03/099316 (Bristol-Myers Squibb), U.S. Pat. No. 6,869,964, WO 03/099274 (Bristol-Myers Squibb), U.S. Pat. No. 6,995,174, WO 2004/032827 (Bristol-Myers Squibb), U.S. Pat. No. 7,041,698, WO 2004/043339 and U.S. Pat. No. 6,878,722 (Bristol-Myers Squibb).

Thiazolidine derivatives which show relevant inhibition in a reverse-phase HPLC assay with an NS3/4A fusion protein and NS5A/5B substrate (Sudo K. et al., Antiviral Research, 1996, 32, 9-18), especially compound RD-1-6250, possessing a fused cinnamoyl moiety substituted with a long alkyl chain, RD4 6205 and RD4 6193;

Thiazolidines and benzanilides identified in Kakiuchi N. et al., J. EBS Letters 421, 217-220; Takeshita N. et al., Analytical Biochemistry, 1997, 247, 242-246;

A phenanthrenequinone possessing activity against protease in a SDS-PAGE and autoradiography assay isolated from the fermentation culture broth of Streptomyces sp., SCH 68631 (Chu M. et al., Tetrahedron Letters, 1996, 37, 7229-7232), and SCH 351633, isolated from the fungus Penicillium griseofulvum, which demonstrates activity in a scintillation proximity assay (Chu M. et al., Bioorganic and Medicinal Chemistry Letters 9, 1949-1952);

Helicase inhibitors (Diana G. D. et al., Compounds, compositions and methods for treatment of hepatitis C, U.S. Pat. No. 5,633,358; Diana G. D. et al., Piperidine derivatives, pharmaceutical compositions thereof and their use in the treatment of hepatitis C, PCT WO 97/36554);

HCV polymerase inhibitors, including nucleoside and non-nucleoside polymerase inhibitors, such as ribavirin, viramidine, clemizole, filibuvir (PF-00868554), HCV POL, NM 283 (valopicitabine), MK-0608, 7-Fluoro-MK-0608, MK-3281, IDX-375, ABT-072, ABT-333, ANA598, BI 207127, GS 9190, PSI-6130, R1626, PSI-6206, PSI-938, PSI-7851, PSI-7977, RG1479, RG7128, HCV-796 VCH-759, VCH-916, INX-189, and GS6620.

Gliotoxin (Ferrari R. et al., Journal of Virology, 1999, 73, 1649-1654), and the natural product cerulenin (Lohmann V. et al., Virology, 1998, 249, 108-118);

Interfering RNA (iRNA) based antivirals, including short interfering RNA (siRNA) based antivirals, such as Sirna-034 and others described in International Patent Publication Nos. WO/03/070750 and WO 2005/012525, and US Patent Publication No. US 2004/0209831.

Antisense phosphorothioate oligodeoxynucleotides (S-ODN) complementary to sequence stretches in the 5′ non-coding region (NCR) of the virus (Alt M. et al., Hepatology, 1995, 22, 707-717), or nucleotides 326-348 comprising the 3′ end of the NCR and nucleotides 371-388 located in the core coding region of the HCV RNA (Alt M. et al., Archives of Virology, 1997, 142, 589-599; Galderisi U. et al., Journal of Cellular Physiology, 1999, 181, 251-257);

Inhibitors of IRES-dependent translation (Ikeda N et al., Agent for the prevention and treatment of hepatitis C, Japanese Patent Pub. JP-08268890; Kai Y. et al., Prevention and treatment of viral diseases, Japanese Patent Pub. JP-10101591);

HCV entry inhibitors, such as celgosivir (MK-3253) (MIGENIX Inc.), SP-30 (Samaritan Pharmaceuticals), ITX4520 (iTherX), ITX5061 (iTherX), PRO-206 (Progenics Pharmaceuticals) and other entry inhibitors by Progenics Pharmaceuticals, e.g., as disclosed in U.S. Patent Publication No. 2006/0198855.

Ribozymes, such as nuclease-resistant ribozymes (Maccjak, D. J. et al., Hepatology 1999, 30, abstract 995) and those disclosed in U.S. Pat. No. 6,043,077 to Barber et al., and U.S. Pat. Nos. 5,869,253 and 5,610,054 to Draper et al.; and

Nucleoside analogs have also been developed for the treatment of Flaviviridae infections.

In certain embodiments, the compounds provided herein, including, by not limited to the pharmaceutical compositions including compound 1 API provided herein, such as those described in paragraphs (A)-(AL), can be administered in combination with any of the compounds described by Idenix Pharmaceuticals in International Publication Nos. WO 01/90121, WO 01/92282, WO 2004/003000, 2004/002422 and WO 2004/002999.

Other patent applications disclosing the use of certain nucleoside analogs that can be used as second therapeutic agents to treat hepatitis C virus include: PCT/CA00/01316 (WO 01/32153; filed Nov. 3, 2000) and PCT/CA01/00197 (WO 01/60315; filed Feb. 19, 2001) filed by BioChem Pharma, Inc. (now Shire Biochem, Inc.); PCT/US02/01531 (WO 02/057425; filed Jan. 18, 2002); PCT/US02/03086 (WO 02/057287; filed Jan. 18, 2002); U.S. Pat. Nos. 7,202,224; 7,125,855; 7,105,499 and 6,777,395 by Merck & Co., Inc.; PCT/EP01/09633 (WO 02/18404; published Aug. 21, 2001); US 2006/0040890; 2005/0038240; 2004/0121980; 6,846,810; 6,784,166 and 6,660,721 by Roche; PCT Publication Nos. WO 01/79246 (filed Apr. 13, 2001), WO 02/32920 (filed Oct. 18, 2001) and WO 02/48165; US 2005/0009737; US 2005/0009737; 7,094,770 and 6,927,291 by Pharmasset, Ltd.

Further compounds that can be used as second therapeutic agents to treat hepatitis C virus are disclosed in PCT Publication No. WO 99/43691 to Emory University, entitled “2′-Fluoronucleosides”. The use of certain 2′-fluoronucleosides to treat HCV is disclosed.

Other miscellaneous compounds that can be used as second therapeutic agents include 1-amino-alkylcyclohexanes (U.S. Pat. No. 6,034,134 to Gold et al.), alkyl lipids (U.S. Pat. No. 5,922,757 to Chojkier et al.), vitamin E and other antioxidants (U.S. Pat. No. 5,922,757 to Chojkier et al.), squalene, amantadine, bile acids (U.S. Pat. No. 5,846,964 to Ozeki et al.), N-(phosphonoacetyl)-L-aspartic acid, (U.S. Pat. No. 5,830,905 to Diana et al.), benzenedicarboxamides (U.S. Pat. No. 5,633,388 to Diana et al.), polyadenylic acid derivatives (U.S. Pat. No. 5,496,546 to Wang et al.), 2′,3′-dideoxyinosine (U.S. Pat. No. 5,026,687 to Yarchoan et al.), benzimidazoles (U.S. Pat. No. 5,891,874 to Colacino et al.), plant extracts (U.S. Pat. No. 5,837,257 to Tsai et al., U.S. Pat. No. 5,725,859 to Omer et al., and U.S. Pat. No. 6,056,961), and piperidenes (U.S. Pat. No. 5,830,905 to Diana et al.).

### Exemplary Second Therapeutic Agents for Treatment of HCV

In certain embodiments, one or more pharmaceutical compositions including compound 1 API provided herein, such as those described in paragraphs (A)-(AL), can be administered in combination or alternation with an anti-hepatitis C virus interferon, such as Intron A® (interferon alfa-2b) and Pegasys® (Peginterferon alfa-2a); Roferon A® (Recombinant interferon alfa-2a), Infergen® (consensus interferon; interferon alfacon-1), PEG-Intron® (pegylated interferon alfa-2b) and Pegasys® (pegylated interferon alfa-2a).

In certain embodiments, the anti-hepatitis C virus interferon is infergen, IL-29 (PEG-Interferon lambda), R7025 (Maxy-alpha), Belerofon, Oral Interferon alpha, BLX-883 (Locteron), omega interferon, multiferon, medusa interferon, Albuferon or REBIF®.

In certain embodiments, one or more pharmaceutical compositions including compound 1 API provided herein, such as those described in paragraphs (A)-(AL), can be administered in combination or alternation with an anti-hepatitis C virus polymerase inhibitor, such as ribavirin, viramidine, HCV POL, NM 283 (valopicitabine), MK-0608, 7-Fluoro-MK-0608, PSI-6130, R1626, PSI-6206, PSI-938, R1479, HCV-796, R7128, PSI-938, PSI-7851, or PSI-7977.

In certain embodiments, the pharmaceutical compositions including compound 1 API provided herein, such as those described in paragraphs (A)-(AL), can be administered in combination with ribavarin and an anti-hepatitis C virus interferon, such as Intron A® (interferon alfa-2b) and Pegasys® (Peginterferon alfa-2a); Roferon A® (Recombinant interferon alfa-2a), Infergen® (consensus interferon; interferon alfacon-1), PEG-Intron® (pegylated interferon alfa-2b) and Pegasys® (pegylated interferon alfa-2a).

In certain embodiments, one or more pharmaceutical compositions including compound 1 API provided herein, such as those described in paragraphs (A)-(AL), can be administered in combination or alternation with an anti-hepatitis C virus protease inhibitor such as ITMN-191, boceprevir (Victrelis™), telaprevir (Incivek™), or Medivir HCV Protease Inhibitor.

In certain embodiments, one or more pharmaceutical compositions including compound 1 API provided herein, such as those described in paragraphs (A)-(AL), can be administered in combination or alternation with an anti-hepatitis C virus vaccine, such as TG4040, PeviPROTM, CGI-5005, HCV/MF59, GV1001, IC41 or INN00101 (E1).

In certain embodiments, one or more pharmaceutical compositions including compound 1 API provided herein, such as those described in paragraphs (A)-(AL), can be administered in combination or alternation with an anti-hepatitis C virus monoclonal antibody, such as AB68 or XTL-6865 (formerly HepX-C); or an anti-hepatitis C virus polyclonal antibody, such as cicavir.

In certain embodiments, one or more pharmaceutical compositions including compound 1 API provided herein, such as those described in paragraphs (A)-(AL), can be administered in combination or alternation with an anti-hepatitis C virus immunomodulator, such as Zadaxin® (thymalfasin), NOV-205 or Oglufanide.

In certain embodiments, one or more pharmaceutical compositions including compound 1 API provided herein, such as those described in paragraphs (A)-(AL), can be administered in combination or alternation with Nexavar, doxorubicin, PI-88, amantadine, JBK-122, VGX-410C, MX-3253 (Ceglosivir), Suvus (BIVN-401 or virostat), PF-03491390 (formerly IDN-6556), G126270, UT-231B, DEBIO-025, EMZ702, ACH-0137171, MitoQ, ANA975, AVI-4065, Bavituxinab (Tarvacin), Alinia (nitrazoxanide) or PYN17.

### EXAMPLES

As used herein, the symbols and conventions used in these processes, schemes and examples, regardless of whether a particular abbreviation is specifically defined, are consistent with those used in the contemporary scientific literature. Specifically, but without limitation, the following abbreviations may be used in the examples and throughout the specification: g (grams); mg (milligrams); ng (nanograms); kg (kilograms); mL (milliliters); μL (microliters); mM (millimolar); μM (micromolar); mmol (millimoles); hr or hrs (hours); min (minutes); μm (micrometer); cps (centipoise); mPa·s (milliPascal·seconds); cc (cubic centimeters, cm³); kN (kilo Newton); MS (mass spectrometry); HPLC (high pressure liquid chromatography); FDA (United States Food and Drug Administration); ICH (International Council on Harmonization of Technical Requirements for Registration of Pharmaceuticals for Human Use); PEG (polyethylene glycol): cGMP (current Good Manufacturing Practice); USP (United States Pharmacopeia), NF (United States National Formulary); GLP (Good Laboratory Practice); PK (pharmacokinetic); Cₘₐₓ (maximum concentration); Cₗₐₛₜ (last plasma concentration); Tₗₐₛₜ (time of last blood draw for plasma assay); t_{1/2} (plasma half-life); and AUC₀₋ₜ (area under the plasma concentration vs. time curve from time 0 (dosing) to time t).

For all of the following examples, standard work-up and purification methods known to those skilled in the art can be utilized. Unless otherwise indicated, all temperatures are expressed in ° C. (degrees Centigrade). All processes and reactions are conducted at room temperature (about 20° C. to about 22° C.) unless otherwise noted.

### Example 1A

### Liquid Filled Capsule Composition

An oral pharmaceutical composition of liquid filled capsules was developed, and manufactured under current Good Manufacturing Practices (cGMP). The following formulation was used:

TABLE 1
Liquid Filled Capsule Composition (Lot #3075833R)

| Ingredients | mg/capsule |
|---|---|
| Compound 1 | 50 |
| Polyethylene Glycol 200 | 201 |
| Polyethylene Glycol 6000 | 100(qs) |
| Sodium Lauryl Sulfate | 15 |
| Capsule fill weight | 366 |
| Capsule shell (theoretical wt.) | 76 |
| Total capsule weight | 442 |

The excipients, Polyethylene Glycol 200, Polyethylene Glycol 6000, and Sodium Lauryl Sulfate complied with the USP/NF monographs. The capsules used were a size 1 hydroxypropyl methylcellulose (HPMC) capsules. The capsules were filled by conventional methods. A stability protocol of the capsules was initiated following standard FDA/ICH guidelines. The following results were obtained:

TABLE 2
Stability Results

| | | | 3 months @ |
|---|---|---|---|
| | Lot Release | 3 months @ | 5° C./Ambient |
| Test | (time 0) | 25° C./60% RH | RH |
| Appearance | Conforms | Conforms | Conforms |
| Identification | Conforms | Conforms | Conforms |
| Assay | 103.6% of | 98.1% Label | 101.5% Label |
| | Label claim | claim | claim |
| Diastereomers | Conforms | Conforms | Conforms |
| 1 & 2 | | | |
| Related | Conforms | Conforms | Conforms |
| Substances | | | |
| Residual | 98 ppm | 34 ppm | 81 ppm |
| Ethylene | | | |
| Sulfide | | | |
| Dissolution | Conforms | Conforms | Conforms |
| Water | Conforms | Conforms | Conforms |
| Determination | | | |

The capsules were used in Phase I and Phase IIa human clinical studies performed in conformance with the Declaration of Helsinki and approved by a local Ethics Committee. Results of the clinical studies indicated an acceptable pharmacokinetic profile.

### Example 2

### Excipient Compatibility Studies

Excipient compatibility of compound 1 with various excipients was determined. Samples of a 1:1 weight ratio of compound 1 and each excipient were blended in glass vials. The vials were subjected to gentle rotation to 30 minutes at ambient conditions. Samples were assayed for ethylene sulfide. Table 3 below presented the results for a selected group of the excipients evaluated:

TABLE 3
Excipient Compatibility Results

| | | ppm Ethylene |
|---|---|---|
| | | Sulfide |
| Sample ID | Excipient Function | Mean of 4 samples |
| Compound 1 | Active | <9 |
| Lactose monohydrate | Filler/Binder | <9 |
| Crospovidone | Disintegrant | <9 |
| Sodium Lauryl Sulfate | Solubilizer | 47 |
| Sodium Stearyl | Lubricant | >438* |
| Fumarate | | |
| Prosolv | Filler/Binder | <9 |
| Stearic Acid | Lubricant | ≦10 |
| PEG8000 | Filler/Lubricant | <10 |
| Poloxamer 188 | Solubilizer/Plasticizer | ≦10 |
| Poloxamer 407 | Solubilizer/Plasticizer | 17 |
| Magnesium Stearate | Lubricant | 389 |
| Aerosil 200 | Glidant | 12 |
| LUTROL MICRO ® 68 | Solubilizer/Plasticizer | 13 |
| LUTROL MICRO ® 127 | Solubilizer/Plasticizer | 18 |
| Gelatin | Capsule | 15 |
| Cellulose, | Filler/Binder | 12 |
| Microcrystalline | | |
| Sodium Starch | Disintegrant | 64 |
| Glycolate | | |
| Calcium Phosphate | Filler/Binder | 27 |
| Dibasic Dihydrate | | |
| Calcium Phosphate | Filler/Binder | 28 |
| Dibasic Anhydrous | | |
| Calcium Phosphate | Filler/Binder | 89 |
| Tribasic | | |
| HPMC | Capsule | 12 |
| Gelatin, Repeat | Capsule | ND |
| Croscarmellose Sodium | Disintegrant | 74 |
| PEG3350 | Filler/Lubricant | <10 |
| Povidone | Binder | <10 |
| Hydroxypropyl Cellulose | Binder | 14 |

*Above the limits of the calibration curve, based on extrapolation

As Table 3 above demonstrates the formation of ethylene sulfide results from the combination of compound 1 with specific excipients. These excipients include magnesium stearate, one of the most commonly used tablet lubricants, as well as sodium stearyl fumarate, another tablet lubricant. Also included are two commonly used tablet disintegrants, croscarmellose sodium and sodium starch glycolate. Several fillers/binders that cause the formation of ethylene sulfide are calcium phosphate dibasic dihydrate, phosphate dibasic anhydrous, and calcium tribasic. In addition, the solubilizer/surfactant sodium lauryl sulphate exhibits a high level of ethylene sulfide when combined with compound 1, and poloxamer 407 and LUTROL MICRO® 127, the BASF trade name for a micronized poloxamer 407, also solubilizers, exhibit unacceptable levels of ethylene sulfide. In summary, many commonly used tablet excipients create a stability problem when combined with compound 1.

Other excipients that were evaluated and which exhibited acceptable levels of ethylene sulfide include: mannitol, magnesium sulphate, talc, castor oil, AEROSIL® 200, pregelatinized starch, microcrystalline cellulose, D-fructose, kaolin, soluble starch, sucrose, hydrogenated vegetable oil, and D-sorbitol.

### Example 3

### Tablet Composition

An oral pharmaceutical composition in the form of a tablet dosage form was developed, and manufactured first as an engineering lot. The following formulation, provided in Table 4, was used:

TABLE 4
Tablet Formulation (Engineering Lot - Lot DM13-047)

| Ingredients | w/w % |
|---|---|
| Compound 1, pin-milled | 27.0 |
| KLUCEL EXF Pharm | 5.00 |
| PROSOLV SMCC 90 | 55.00 |
| KOLLIDON CL | 5.00 |
| LUTROL 68 MICRO | 5.00 |
| AEROSIL R972 | 1.00 |
| Stearic acid | 2.00 |
| Total | 100.00 |

The compound 1 was pin-milled to obtain an average particle size in the range of 200 to 300 μm as determined by Dynamic Light Scattering. KLUCEL® EXF Pharm is a pharmaceutical grade of hydroxypropyl cellulose sold by Ashland, Inc. A 10% (by weight) solution of KLUCEL® EXF in water has a viscosity in the range of 300 to 600 cps when measured at 25° C. using a Brookfield LVF, LVDV-1+, or LVDV-E viscometer, spindle 2, speed 30 rpm. The “X” refers to the fine grind particle size in which a minimum of 99.9% of the particles (by weight) pass through a U.S. 60 mesh sieve screen, a minimum of 90% pass through a U.S. 80 mesh sieve screen, and a minimum of 80% passing through a U.S. 100 mesh sieve screen. PROSOLV SMCC® 90, manufactured by JRS Pharma, is a silicified microcrystalline cellulose. PROSOLV SMCC® 90 complies with the United States NF monographs, and the vendor reports that PROSOLV SMCC® 90 has an average particle size of about 110 μm as determined by laser diffraction. PROSOLV SMCC® 90 is specifically designed for direct compression formulations. KOLLIDON® CL is manufactured by BASF and complies with the United States NF monograph for crospovidone. KOLLIDON® CL is the standard particle size grade, and the vendor reports an average particle size of 110 to 130 μm. LUTROL® 68 MICRO is manufactured by BASF, and is the trade name for poloxamer 188. LUTROL® 68 MICRO is a micronized block copolymer of polyethylene oxide-polypropylene oxide with an average particle size of about 50 μm as determined by Malvern Mastersize 2000, with a specification that not more than 50% by weight is retained on a #270 sieve screen (53 μm screen) using Alpine Air Jet Sieve Analysis. The weight average molecular weight of LUTROL® 68 MICRO is from about 7680 to 9510 g/mol. AEROSIL® R972 is hydrophobic colloidal silica, and complies with the United States NF monograph. AEROSIL R972 has a BET surface area of about 110±m²/gram. The stearic acid utilized was supplied by Mallinckrodt as stearic acid, powder, 2216.

The % “active pharmaceutical ingredient” in the above formulation is adjusted to obtain 50 mg of compound 1 per 200 mg tablet. In the present case, the potency of the active pharmaceutical ingredient was 92.6%. If the active were to exhibit a different potency, the amount of the PROSOLV SMCC® 90, the filler, would be adjusted as necessary to maintain a 200 mg tablet weight while maintaining the weight percent of the other excipients the same as above.

Tablets were manufactured at a batch size of 400 g. AEROSIL R972 and a portion of the PROSOLV SMCC® 90 were pre-blended and passed through a 20 mesh screen. All remaining excipients and the active pharmaceutical ingredient, compound 1, pin-milled, were passed through a 20 mesh screen prior to being blended in a 2 Quart Patterson-Kelly V-Blender. The blend was then directly compressed on a Korsch XL100 PRO® tablet press with a gravity feeder using standard concave 0.3125″ round tooling (Thomas Engineering, Hoffman Estates, Ill.) at a pressure of 7.1±kN to obtain an average tablet hardness of 11.9 kP (range 11.1 kP to 13.0 kP). Tablet hardness was determined using a tablet hardness tester as per USP general chapter <1217>. Tablet flow was found to be acceptable.

Using a validated HPLC assay method, the tablets were assayed. The tablets assayed at 101.4% of label claim of compound 1, and the content uniformity was 105.7% with an RSD of 2.7%. The friability was less than 0.1%. The disintegration was acceptable with a mean disintegration time of 6.7 minutes and a minimum of 6.0 min and a maximum of 7.9 min obtained using a disintegration tester as per USP general chapter <701>. The dissolution was determined using USP apparatus II (paddle) at 75 rpm, 1000 ml of dissolution media of 4.5 acetate buffer (per USP) with 1% (by weight) sodium lauryl sulphate using 6 tablets. The dissolution measurements were 86.9%, 95.7%, 98.3%, and 99.4% of label claim at the 15 min, 30 min, 45 min, and 60 min time points, respectively. Microbial limits as per USP chapters <61> and <62> were met for the tablets at lot release. The % of diastereomers 1 and 2 was each within the 45% to 55% range.

### Example 4

### Tablet Pharmacokinetics in Monkey

A lot of tablets manufactured according to the formula of Table 4 except that the Compound 1, pin-milled was 25.75% by weight of the formulation, and the PROSOLV SMCC 90 was 56.25% by weight of the formulation. In addition, the tablets had a 25-mg dose of Compound 1 and were compressed to a 100-mg weight at 4.5 kN using 0.25″ round standard concave tooling. A single dose pharmacokinetic study approved by the Animal Care and Use Committee was conducted in 4 male cynomolgous monkeys in the fasted state. The tablet formulation was compared to the clinical trial material (CTM) capsule (formulation not provided here) at a 25-mg dose of Compound 1.

The pharmacokinetic samples were assayed for Compound 1 as well as 2′-C-methyl-guanosine. Data from the pharmacokinetic study in monkeys are presented in Tables 5-8 below:

TABLE 5
Individual pharmacokinetic parameters of Compound 1, Tablet Formulation

| | | | | | | | | AUCₗₐₛₜ/ |
|---|---|---|---|---|---|---|---|---|
| | | | | Cₘₐₓ/ | | | | Dose^{a} |
| | Dose | | Cₘₐₓ | Dose^{a} | | | AUCₗₐₛₜ | (hr*ng/ |
| Animal | (mg/ | Tₘₐₓ | (ng/ | (ng/mL)/ | Tₗₐₛₜ | Cₗₐₛₜ | (hr*ng/ | mL)/ |
| ID | kg) | (hr) | mL) | (mg/kg) | (hr) | (ng/mL) | mL) | (mg/kg) |
| A | 7.23 | 6 | 0.742 | 0.10 | 12 | 0.152 | 2.73 | 0.38 |
| B | 7.79 | 8 | 0.831 | 0.11 | 12 | 0.173 | 4.43 | 0.57 |
| C | 7.25 | 8 | 0.25 | 0.034 | 12 | 0.169 | 1.7 | 0.23 |
| D | 7.67 | 6 | 0.654 | 0.085 | 12 | 0.113 | 2.72 | 0.36 |
| | Mean | 6-8 | 0.619 | 0.082 | 12 | 0.152 | 2.9 | 0.38 |
| | SD | | 0.257 | 0.033 | | 0.027 | 1.13 | 0.14 |

^{a}Normalized to a dose of 1 mg/kg

TABLE 6
Individual pharmacokinetic parameters of 2′-C-methylguanosine,
Tablet Formulation

| | | | | | | | | | | AUC₀₋₂₄/ |
|---|---|---|---|---|---|---|---|---|---|---|
| | Dose | | | Cₘₐₓ | Cₘₐₓ/Dose^{a} | | Cₗₐₛₜ | AUCₗₐₛₜ | AUC₀₋₂₄ | Dose^{a} |
| Animal | (mg/ | t_{1/2} | Tₘₐₓ | (ng/ | (ng/mL)/ | Tₗₐₛₜ | (ng/ | (hr*ng/ | (hr*ng/ | (hr*ng/mL)/ |
| ID | kg) | (hr) | (hr) | mL) | (mg/kg) | (hr) | mL) | mL) | mL) | (mg/kg) |
| A | 7.23 | 7.2 | 6 | 10.6 | 1.5 | 36 | 0.551 | 140 | 121 | 17 |
| B | 7.79 | 19 | 8 | 17.1 | 2.2 | 48 | 1.26 | 215 | 159 | 20 |
| C | 7.25 | 9.4 | 8 | 7.50 | 1.0 | 36 | 1.23 | 157 | 127 | 18 |
| D | 7.67 | 8.8 | 6 | 21.3 | 2.8 | 48 | 0.799 | 280 | 215 | 28 |
| | Mean | 11 | 6-8 | 14.1 | 1.9 | 36- | 0.96 | 198 | 156 | 21 |
| | SD | 5.3 | | 6.24 | 0.77 | 48 | 0.345 | 63.5 | 43.0 | 5.2 |

^{a}Normalized to a dose of 1 mg/kg

TABLE 7
Individual pharmacokinetic parameters of Compound 1, CTM capsule

| | | | | Cₘₐₓ/Dose^{a} | | | | AUCₗₐₛₜ/Dose^{a} |
|---|---|---|---|---|---|---|---|---|
| Animal | Dose | Tₘₐₓ | Cₘₐₓ | (ng/mL)/ | Tₗₐₛₜ | Cₗₐₛₜ | AUCₗₐₛₜ | (hr*ng/mL)/ |
| ID | (mg/kg) | (hr) | (ng/mL) | (mg/kg) | (hr) | (ng/mL) | (hr*ng/mL) | (mg/kg) |
| E | 8.17 | 0.5 | 3.21 | 0.39 | 12 | 0.369 | 7.91 | 0.97 |
| F | 8.36 | 0.5 | 1.02 | 0.12 | 8 | 0.209 | 2.56 | 0.31 |
| G | 8.17 | 12 | 0.633 | 0.077 | 12 | 0.633 | 3.36 | 0.41 |
| H | 8.25 | 6 | 0.446 | 0.054 | 12 | 0.111 | 1.75 | 0.21 |
| | Mean | 0.5- | 1.33 | 0.16 | 8- | 0.331 | 3.89 | 0.47 |
| | SD | 12 | 1.28 | 0.16 | 12 | 0.228 | 2.76 | 0.34 |

^{a}Normalized to a dose of 1 mg/kg

TABLE 8
Individual pharmacokinetic parameters of 2′-C-methylguanosine,
CTM capsule

| | | | | | | | | | | AUC₀₋₂₄/ |
|---|---|---|---|---|---|---|---|---|---|---|
| | | | | | Cₘₐₓ/Dose^{a} | | Cₗₐₛₜ | AUCₗₐₛₜ | AUC₀₋₂₄ | Dose |
| Animal | Dose | t_{1/2} | Tₘₐₓ | Cₘₐₓ | (ng/mL)/ | Tₗₐₛₜ | (ng/ | (hr*ng/ | (hr*ng/ | (hr*ng/mL)/ |
| ID | (mg/kg) | (hr) | (hr) | (ng/mL) | (mg/kg) | (hr) | mL) | mL) | mL) | (mg/kg) |
| E | 8.17 | 7.15 | 1 | 13.6 | 1.7 | 36 | 0.511 | 166 | 148 | 18 |
| F | 8.36 | 5.5 | 6 | 12.8 | 1.5 | 24 | 1 | 123 | 123 | 15 |
| G | 8.17 | 8.7 | 6 | 4.87 | 0.60 | 24 | 1.16 | 68.4 | 68.4 | 8.4 |
| H | 8.25 | 9.55 | 6 | 10.8 | 1.3 | 36 | 1.42 | 189 | 160 | 19 |
| | Mean | 7.7 | 1-6 | 10.5 | 1.3 | 24- | 1.02 | 137 | 125 | 15 |
| | SD | 1.8 | | 3.94 | 0.5 | 36 | 0.38 | 53.1 | 40.7 | 4.9 |

^{a}Normalized to a dose of 1 mg/kg

The exposure of Compound 1 was low and variable for both formulations. The time to reach the maximum observed concentration, Tₘₐₓ, was longer and less variable for the tablet compared to the CTM. Dose normalized exposures to Compound 1 were slightly higher for the capsule, by 2-fold for Cₘₐₓ/D and by 1.2-fold for AUCₗₐₛₜ/D values.

The pharmacokinetic parameters for 2′-C-methyl-guanosine were overall similar for the two formulations. Tₘₐₓ was similar but appeared less variable for the tablet compared to the CTM. Dose normalized exposures to 2′-C-methyl-guanosine were slightly higher for the tablet, by 1.5-fold for Cₘₐₓ/D and by 1.4-fold for AUCₗₐₛₜ/D values.

### Example 5

### Tablet Pharmacokinetics in Man

A lot of tablets manufactured under cGMP conditions utilizing the composition of Example 3 (with appropriate adjustment of the weight % of PROSOLV SMCC 90 to account for the potency of Compound 1) were utilized in a pharmacokinetic study. The human clinical study was study performed in conformance with the Declaration of Helsinki and approved by a local Ethics Committee. An open-label randomized cross-over single-dose pharmacokinetic effect study was conducted in 12 healthy male subjects (ages 19-65) to compare the relative exposure of the tablet composition (50 mg compound 1) as compared to the liquid filled capsule composition (50 mg of compound 1) in the fasted state. In addition, an un-randomized third period followed the first two periods in which all 12 subjects were dosed with the tablet composition (50 mg compound 1) in the fed state, and 8 of the 12 subjects returned for a fourth period in which the 8 subjects were dosed with the liquid filled capsule composition (50 mg compound 1) in the fed state. Plasma samples for pharmacokinetic (PK) analysis were taken over a period of 120 h after dosing during each treatment period. Dosing days were at least 6 days apart (Period 1, days 1 to 6; Period 2, days 7 to 13, Period 3, days 14 to 20; and Period 4 followed the initial three periods).

No serious adverse events were reported. The pharmacokinetic samples were assayed for 2′-C-methyl-guanosine as well as compound 1. Relative exposure of the tablet composition under fed conditions is summarized in Table 9 below.

TABLE 9
Relative Exposure under fed conditions: Tablet vs. Capsule (N = 8)

| | | | |
|---|---|---|---|
| | Geometric | Geometric | |
| Parameter | mean Capsule | mean Tablet | Ratio (90% CI) |

Compound 1

| | | | |
|---|---|---|---|
| Cₘₐₓ (ng/mL) | 3.0 | 2.4 | 77.26 (56.43, 105.8) |
| AUC₀₋ₜ (ng/mL*hr) | 8.7 | 6.7 | 77.19 (62.12, 95.92) |

2′-C-methyl-guanosine

| | | | |
|---|---|---|---|
| Cₘₐₓ (ng/mL) | 13.2 | 11.2 | 84.76 (60.80, 118.2) |
| AUC₀₋ₜ (ng/mL*hr) | 227 | 246 | 108.3 (95.64, 122.5) |

The relative exposure of the tablet vs. the capsule was 84.8% and 108.3% based on the Cₘₐₓ and AUC₀₋ₜ of 2′-C-methyl-guanosine (2′-MeG), respectively. Moreover, the relative bioavailability based on the 2′-MeG AUC₀₋ₜ met the criteria for bioequivalence. The Cₘₐₓ and AUC₀₋ₜ of compound 1 associated with the tablet were 77.3% and 77.2% of the respective values of the capsule.

The food effect on the pharmacokinetics of the tablet composition is summarized in Table 10 below.

TABLE 10
Food effect on the tablet (N = 12)

| | | | |
|---|---|---|---|
| | Geometric | Geometric | |
| Parameter | mean Fasted | mean Fed | Ratio (90% CI) |

Compound 1

| | | | |
|---|---|---|---|
| Cₘₐₓ (ng/mL) | 6.3 | 2.4 | 37.08 (29.91, 45.96) |
| AUC₀₋ₜ (ng/mL*hr) | 12.8 | 6.7 | 52.15 (44.37, 61.29) |

2′-C-methyl-guanosine

| | | | |
|---|---|---|---|
| Cₘₐₓ (ng/mL) | 17.2 | 11.2 | 64.84 (48.10, 87.40) |
| AUC₀₋ₜ (ng/mL*hr) | 327 | 246 | 75.28 (65.75, 86.18) |

The Cₘₐₓ and AUC₀₋ₜ of compound 1 associated with the tablet under fed conditions were 37.1% and 52.2% of the respective values obtained under fasted conditions. The Cₘₐₓ and AUC₀₋ₜ of 2′-MeG associated with the tablet under fed conditions were 64.8% and 75.3% of the respective values under fasted conditions (see Table 10, above). The plasma exposures of the tablet dosed under fed conditions were about 40-50% lower for IDX184 and 25-35% lower for 2′-MeG compared to the capsule under fasted conditions.

Overall, under fed conditions, the 50 mg tablet delivered similar plasma exposures of 2′-MeG and slightly lower exposures of compound 1 when compared to the capsule. Statistical results indicate that based on the total plasma exposure of 2′-MeG, the two formulations were bioequivalent under fed conditions. Dosing with the tablet under fasted conditions as compared to fed conditions increased exposure to compound 1 and to 2′-MeG. This food effect was less for the tablet formulation when compared to the liquid filled capsule formulation. Since 2′-MeG is the target nucleoside and compound 1 is expected to be dosed with food, relative bioavailability based on plasma 2′-MeG obtained under fed conditions is more clinically relevant.

### Example 6

### Tablet Stability

Another lot of tablets, 200 mg tablet weight containing a 50 mg dose of compound 1, utilizing the composition of Example 3 was utilized for stability. The tablets were packaged in 30 cc High Density Polyethylene (HDPE) bottles with a 28 mm child resistant closure lined with aluminum foil/polypropylene that is sealed by induction seal. A 0.5 g silica gel desiccant was included in each bottle. A stability protocol of the tablets was initiated following standard FDA/ICH guidelines. The same test methods were used in the stability protocol as were used in the lot release testing. The following results were obtained:

TABLE 11
Stability Results Tablets (Lot 3282-01) up to 3 months

| | | | 3 months @ |
|---|---|---|---|
| | Lot Release | 3 months @ | 5° C./Ambient |
| Test | 3282-01(time 0) | 25° C./60% RH | RH |
| Appearance | Conforms | Conforms | Conforms |
| Identification | Conforms | Conforms | Conforms |
| Assay | 102.3% label | 97.8% label | 95.1% label |
| | claim | claim | claim |
| Diastereomers | Conforms | Conforms | Conforms |
| 1 & 2 | | | |
| Related | Conforms | Conforms | Conforms |
| Substances | | | |
| Residual | Not detected | Not detected | Not detected |
| Ethylene | | | |
| Sulfide | | | |
| Dissolution | 102% in 60 min | 97% at 60 min | 100% at 60 min |
| Water | Conforms | Conforms | Conforms |
| Determination | | | |

TABLE 12
Stability Results Tablets (Lot 3282-01) at 9 months

| | | | 9 months @ |
|---|---|---|---|
| | Lot Release | 9 months @ | 5° C./Ambient |
| Test | 3282-01(time 0) | 25° C./60% RH | RH |
| Appearance | Conforms | Conforms | Conforms |
| Identification | Conforms | Conforms | Conforms |
| Assay | 102.3% label | 96.2% label | 96.1% label |
| | claim | claim | claim |
| Diastereomers | Conforms | Conforms | Conforms |
| 1 & 2 | | | |
| Related | Conforms | Conforms | Conforms |
| Substances | | | |
| Residual | Not detected | Not detected | Not detected |
| Ethylene | | | |
| Sulfide | | | |
| Dissolution | 102% in 60 min | 96% at 60 min | 94% at 60 min |
| Water | Conforms | Conforms | Conforms |
| Determination | | | |

TABLE 13
Stability Results Tablets (Lot 3282-01) at 12 months

| | | | 12 months @ |
|---|---|---|---|
| | Lot Release | 12 months @ | 5° C./Ambient |
| Test | 3282-01(time 0) | 25° C./60% RH | RH |
| Appearance | Conforms | Conforms | Conforms |
| Identification | Conforms | Conforms | Conforms |
| Assay | 102.3% label | 96.5% label | 95.9% label |
| | claim | claim | claim |
| Diastereomers | Conforms | Conforms | Conforms |
| 1 & 2 | | | |
| Related | Conforms | Conforms | Conforms |
| Substances | | | |
| Residual | Not detected | Not detected | Not detected |
| Ethylene | | | |
| Sulfide | | | |
| Dissolution | 102% in 60 min | 94% at 60 min | 95% at 60 min |
| Water | Conforms | Conforms | Conforms |
| Determination | | | |

As shown in Tables 11 and 12 above, no ethylene sulfide was detected at the 9 month time point for the 25° C. condition and for the 9 month time point for the refrigerated (5° C.) condition. In addition, as shown in Table 13, no ethylene sulfide was detected at the 12 month time point for both the 25° C. condition and the refrigerated condition. The method of detecting ethylene sulfide has a limit of detection (LOD) of 1 ppm, a calibrated limit of quantitation (LOQ) of 10 ppm, and a calculated LOQ of 3 ppm. In summary, the tablet formulation is a manufacturable, bioavailable composition that meets all release criteria, and is unexpectedly stable showing no detectable ethylene sulfide after 9 months @ 25° C./60% RH.

All publications, patents, and patent applications cited in this specification are herein incorporated by reference as if each individual publication, patent, or patent application were specifically and individually indicated to be incorporated by reference. While the claimed subject matter has been described in terms of various embodiments, the skilled artisan will appreciate that various modifications, substitutions, omissions, and changes may be made without departing from the spirit thereof. Accordingly, it is intended that the scope of the claimed subject matter is limited solely by the scope of the following claims, including equivalents thereof.

What is claimed is:

1) An oral pharmaceutical composition comprising
an active pharmaceutical ingredient, the active pharmaceutical ingredient being one or more members of the group consisting of the compound of the formula 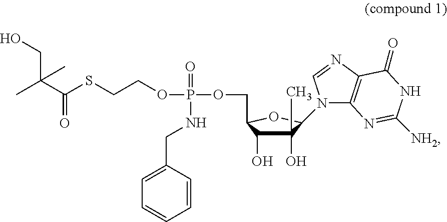
its pharmaceutically acceptable salts, its tautomers, its solvates, and its stereoisomers;
and
one or more members of the group consisting of pharmaceutically acceptable excipients, carriers, and diluents;
wherein when the oral pharmaceutical composition is stored at 25° C. and 60% relative humidity for a duration of time of at least 12 months, the oral pharmaceutical composition comprises not more than 15 μg of ethylene sulfide per each gram of compound 1 in the composition.

2) The oral pharmaceutical composition of claim 1, wherein the duration is 18 months.

3) The oral pharmaceutical composition of claim 1, wherein the duration is 24 months.

4) The oral pharmaceutical composition of claim 1, wherein when stored at 25° C. and 60% relative humidity for a duration of time of at least 12 months, the composition comprises not more than 13 μg of ethylene sulfide per each gram of compound 1 in the composition.

5) The oral pharmaceutical composition of claim 4, wherein when stored at 25° C. and 60% relative humidity for a duration of time of at least 12 months, the composition comprises not more than 10 μg of ethylene sulfide per each gram of compound 1 in the composition.

6) The oral pharmaceutical composition of claim 5, wherein when stored at 25° C. and 60% relative humidity for a duration of time of at least 12 months, the composition comprises not more than 8 μg of ethylene sulfide per each gram of compound 1 in the composition.

7) The oral pharmaceutical composition of claim 6, wherein when stored at 25° C. and 60% relative humidity for a duration of time of at least 12 months, the composition comprises not more than 5 μg of ethylene sulfide per each gram of compound 1 in the composition.

8) The solid oral pharmaceutical composition of claim 1, wherein the oral pharmaceutical composition is a solid oral pharmaceutical composition.

9) The solid oral pharmaceutical composition of claim 8, wherein the solid oral pharmaceutical composition is selected from the group consisting of capsules and tablets.

10) The solid oral pharmaceutical composition of claim 9, wherein the solid oral pharmaceutical composition is a tablet.

11) The tablet of claim 10, wherein the tablet is free from, or essentially free from, sodium lauryl sulfate, sodium stearyl fumarate, poloxamer 407, magnesium stearate, calcium phosphate dibasic dihydrate, phosphate dibasic anhydrous, calcium tribasic, sodium starch glycolate, and croscarmellose sodium.

12) The tablet of claim 11 comprising a disintegrant, a binder, and a tablet lubricant.

13) The tablet of claim 12, further comprising a flow aid.

14) The tablet of claim 13, wherein the tablet comprises crospovidone, hydrophobic colloidal silica, hydroxypropyl cellulose, poloxamer 188, silicified microcrystalline cellulose, and stearic acid.

15) The tablet of claim 14, wherein the tablet, exclusive of any exterior coating, consists essentially of active pharmaceutical ingredient, crospovidone, hydrophobic colloidal silica, hydroxypropyl cellulose, poloxamer 188, silicified microcrystalline cellulose, and stearic acid.

16) The tablet of claim 14, wherein at least 95.0 weight % of the tablet, exclusive of any exterior coating, consists of active pharmaceutical ingredient, crospovidone, hydrophobic colloidal silica, hydroxypropyl cellulose, poloxamer 188, silicified microcrystalline cellulose, and stearic acid.

17) The oral pharmaceutical composition of claim 1, wherein the oral pharmaceutical composition, exclusive of any exterior coating, consists essentially of active pharmaceutical ingredient, crospovidone, hydrophobic colloidal silica, hydroxypropyl cellulose, poloxamer 188, silicified microcrystalline cellulose, and stearic acid.

18) The tablet of claim 14, wherein the tablet, exclusive of any exterior coating, comprises 25.0%±3.0% active pharmaceutical ingredient, 5.0%±2.0% crospovidone, 1.0%±0.5% hydrophobic colloidal silica, 5.0%±2.5% hydroxypropyl cellulose, 5.0%±2.0% poloxamer 188, 55.0%±7.0% silicified microcrystalline cellulose, and 2.0%±1.0% stearic acid; subject to the condition that the total does not exceed 100%, and wherein the % represents weight %.

19) The oral pharmaceutical composition of claim 17, wherein the oral pharmaceutical composition, exclusive of any exterior coating, comprises 25.0%±3.0% active pharmaceutical ingredient, 5.0%±2.0% crospovidone, 1.0%±0.5% hydrophobic colloidal silica, 5.0%±2.5% hydroxypropyl cellulose, 5.0%±2.0% poloxamer 188, 55.0%±7.0% silicified microcrystalline cellulose, and 2.0%±1.0% stearic acid; subject to the condition that the total does not exceed 100%, and wherein the % represents weight %.
